(19) United States
(12) Patent Application Publication
(10) Pub. No.: US 2009/0069336 A1
(43) Pub. Date: Mar. 12, 2009

(54) PYRIMIDOTHIOPHENE COMPOUNDS

(75) Inventors: Xavier Barril-Alonso, Cambridge (GB); Paul Andrew Brough, Cambridge (GB); Martin James Drysdale, Cambridge (GB); Paul Webb, Cambridge (GB)

Correspondence Address: BANNER & WITCOFF, LTD., 1100 13th STREET, N.W., SUITE 1200, WASHINGTON, DC, 20005-4051, US

(73) Assignees: Brian William DYMOCK, Oxon (GB); VERNALIS (CAMBRIDGE) LIMITED., Cambridge (GB); CANCER RESEARCH TECHNOLOGY LTD., London (GB); THE INSTITUTE OF CANCER RESEARCH, London (GB)

(21) Appl. No.: 11/632,969

(22) PCT Filed: Jul. 18, 2005
(86) PCT No.: PCT/GB2005/002816

§ 371 Date: Nov. 19, 2008

(30) Foreign Application Priority Data
Jul. 20, 2004 (GB) 0416168.3

(51) Int. Cl. A61K 31/519; C07D 495/04; A61K 31/496; A61P 35/00; A61P 31/12; A61P 17/00; A61P 3/00

(52) U.S. Cl. 514/252.16; 544/278; 514/260.1

(57) ABSTRACT

Compounds of formula (I) are inhibitors of HSP90, and useful in the treatment of, for example, cancers:

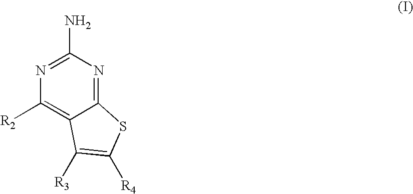

wherein R₂ is a group of formula (IA):

-(Ar¹)ₘ-(Alk¹)ₚ-(Z)ᵣ-(Alk²)ₛ-Q (IA)

wherein in any compatible combination Ar¹ is an optionally substituted aryl or heteroaryl radical, Alk¹ and Alk² are optionally substituted divalent C₁-C₃ alkylene or C₂-C₃ alkenylene radicals, m, p, r and s are independently 0 or 1, Z is —O—, —S—, —(C═O)—, —(C═S)—, —SO₂—, —C(═O)O—, —C(═O)NR^{A}—, —C(═S)NR^{A}—, —SO₂NR^{A}—, —NR^{A}C(═O)—, —NR^{A}SO₂— or —NR^{A}— wherein R^{A} is hydrogen or C₁-C₆ alkyl, and Q is hydrogen or an optionally substituted carbocyclic or heterocyclic radical; R₃ is hydrogen, an optional substituent, or an optionally substituted (C₁-C₆)alkyl, aryl or heteroaryl radical; and R₄ is (i) hydrogen, a —CN group, a nitro group —NO₂, or a —C(═NOH)(NH₂) group, or (ii) an optionally substituted C₁-C₆alkyl, aryl, heterocyclic, aryl(C₁-C₆alkyl)-, or heterocyclic(C₁-C₆alkyl)- group, or (iii) a group of formula —C(═O)R₅ wherein R₅ is hydroxyl, optionally substituted C₁-C₆alkyl, C₁-C₆alkyoxy, aryl, aryloxy, heteroaryl, heteroaryloxy, aryl(C₁-C₆alkyl)-, aryl(C₁-C₆alkoxy)-, heteroaryl(C₁-C₆alkyl)-, or heteroaryl(C₁-C₆alkoxy)-, or (iv) a group of formula —C(═O)NHR₆ wherein R₆ is primary, secondary, tertiary or cyclic amino, or hydroxyl, optionally substituted C₁-C₆alkyl, C₁-C₆alkyoxy, aryl, aryloxy, heteroaryl, heteroaryloxy, aryl(C₁-C₆alkyl)-, aryl(C₁-C₆alkoxy)-, heteroaryl(C₁-C₆alkyl)-, or heteroaryl(C₁-C₆alkoxy)-.

This invention relates to substituted bicyclic thieno[2,3-d]pyrimidine (herein referred to as ‘pyrimidothiophene’) compounds having HSP90 inhibitory activity, to the use of such compounds in medicine, in relation to diseases which are responsive to inhibition of HSP90 activity such as cancers, and to pharmaceutical compositions containing such compounds.

### BACKGROUND TO THE INVENTION

Molecular chaperones maintain the appropriate folding and conformation of proteins and are crucial in regulating the balance between protein synthesis and degradation. They have been shown to be important in regulating many important cellular functions, such as cell proliferation and apoptosis (Jolly and Morimoto, 2000; Smith et al., 1998; Smith, 2001).

### Heat Shock Proteins (HSPs)

Exposure of cells to a number of environmental stresses, including heat shock, alcohols, heavy metals and oxidative stress, results in the cellular accumulation of a number of chaperones, commonly known as heat shock proteins (HSPs). Induction of HSPs protects the cell against the initial stress insult, enhances recovery and leads to maintenance of a stress tolerant state. It has also become clear, however, that certain HSPs may also play a major molecular chaperone role under normal, stress-free conditions by regulating the correct folding, degradation, localization and function of a growing list of important cellular proteins.

A number of multigene families of HSPs exist, with individual gene products varying in cellular expression, function and localization. They are classified according to molecular weight, e.g., HSP70, HSP90, and HSP27.

Several diseases in humans can be acquired as a result of protein misfolding (reviewed in Tytell et al., 2001; Smith et al., 1998). Hence the development of therapies which disrupt the molecular chaperone machinery may prove to be beneficial. In some conditions (e.g., Alzheimer's disease, prion diseases and Huntington's disease), misfolded proteins can cause protein aggregation resulting in neurodegenerative disorders. Also, misfolded proteins may result in loss of wild type protein function, leading to deregulated molecular and physiological functions in the cell.

HSPs have also been implicated in cancer. For example, there is evidence of differential expression of HSPs which may relate to the stage of tumour progression (Martin et al., 2000; Conroy et al., 1996; Kawanishi et al., 1999; Jameel et al., 1992; Hoang et al., 2000; Lebeau et al., 1991). As a result of the involvement of HSP90 in various critical oncogenic pathways and the discovery that certain natural products with anticancer activity are targeting this molecular chaperone, the fascinating new concept has been developed that inhibiting HSP function may be useful in the treatment of cancer. The first molecular chaperone inhibitor is currently undergoing clinical trials.

### HSP90

HSP90 constitutes about 1-2% of total cellular protein, and is usually present in the cell as a dimer in association with one of a number of other proteins (see, e.g., Pratt, 1997). It is essential for cell viability and it exhibits dual chaperone functions (Young et al., 2001). It plays a key role in the cellular stress response by interacting with many proteins after their native conformation has been altered by various environmental stresses, such as heat shock, ensuring adequate protein folding and preventing non-specific aggregation (Smith et al., 1998). In addition, recent results suggest that HSP90 may also play a role in buffering against the effects of mutation, presumably by correcting the inappropriate folding of mutant proteins (Rutherford and Lindquist, 1998). However, HSP90 also has an important regulatory role. Under normal physiological conditions, together with its endoplasmic reticulum homologue GRP94, HSP90 plays a housekeeping role in the cell, maintaining the conformational stability and maturation of several key client proteins. These can be subdivided into three groups: (a) steroid hormone receptors, (b) Ser/Thr or tyrosine kinases (e.g., ERBB2, RAF-1, CDK4, and LCK), and (c) a collection of apparently unrelated proteins, e.g., mutant p53 and the catalytic subunit of telomerase hTERT. All of these proteins play key regulatory roles in many physiological and biochemical processes in the cell. New HSP90 client proteins are continuously being identified.

The highly conserved HSP90 family in humans consists of four genes, namely the cytosolic HSP90α and HSP90β isoforms (Hickey et al., 1989), GRP94 in the endoplasmic reticulum (Argon et al., 1999) and HSP75/TRAP1 in the mitochondrial matrix (Felts et al., 2000). It is thought that all the family members have a similar mode of action, but bind to different client proteins depending on their localization within the cell. For example, ERBB2 is known to be a specific client protein of GRP94 (Argon et al., 1999) and type 1 tumour necrosis factor receptor (TNFR1) and RB have both been shown to be clients of TRAP1 (Song et al., 1995; Chen et al., 1996).

HSP90 participates in a series of complex interactions with a range of client and regulatory proteins (Smith, 2001). Although the precise molecular details remain to be elucidated, biochemical and X-ray crystallographic studies (Prodromou et al., 1997; Stebbins et al., 1997) carried out over the last few years have provided increasingly detailed insights into the chaperone function of HSP90.

Following earlier controversy on this issue, it is now clear that HSP90 is an ATP-dependent molecular chaperone (Prodromou et al, 1997), with dimerization of the nucleotide binding domains being essential for ATP hydrolysis, which is in turn essential for chaperone function (Prodromou et al, 2000a). Binding of ATP results in the formation of a toroidal dimer structure in which the N terminal domains are brought into closer contact with each other resulting in a conformational switch known as the ‘clamp mechanism’ (Prodromou and Pearl, 2000b).

### Known HSP90 Inhibitors

The first class of HSP90 inhibitors to be discovered was the benzoquinone ansamycin class, which includes the compounds herbimycin A and geldanamycin. They were shown to reverse the malignant phenotype of fibroblasts transformed by the v-Src oncogene (Uehara et al., 1985), and subsequently to exhibit potent antitumour activity in both in vitro (Schulte et al., 1998) and in vivo animal models (Supko et al., 1995).

Immunoprecipitation and affinity matrix studies have shown that the major mechanism of action of geldanamycin involves binding to HSP90 (Whitesell et al., 1994; Schulte and Neckers, 1998). Moreover, X-ray crystallographic studies have shown that geldanamycin competes at the ATP binding site and inhibits the intrinsic ATPase activity of HSP90 (Prodromou et al., 1997; Panaretou et al., 1998). This in turn prevents the formation of mature multimeric HSP90 complexes capable of chaperoning client proteins. As a result, the client proteins are targeted for degradation via the ubiquitin proteasome pathway. 17-Allylamino, 17-demethoxygeldanamycin (17AAG) retains the property of HSP90 inhibition resulting in client protein depletion and antitumour activity in cell culture and xenograft models (Schulte et al, 1998; Kelland et al, 1999), but has significantly less hepatotoxicity than geldanamycin (Page et al, 1997). 17AAG is currently being evaluated in Phase I clinical trials.

Radicicol is a macrocyclic antibiotic shown to reverse the malignant phenotype of v-Src and v-Ha-Ras transformed fibroblasts (Kwon et al, 1992; Zhao et al, 1995). It was shown to degrade a number of signalling proteins as a consequence of HSP90 inhibition (Schulte et al., 1998). X-ray crystallographic data confirmed that radicicol also binds to the N terminal domain of HSP90 and inhibits the intrinsic ATPase activity (Roe et al., 1998). Radicicol lacks antitumour activity in vivo due to the unstable chemical nature of the compound.

Coumarin antibiotics are known to bind to bacterial DNA gyrase at an ATP binding site homologous to that of the HSP90. The coumarin, novobiocin, was shown to bind to the carboxy terminus of HSP90, i.e., at a different site to that occupied by the benzoquinone ansamycins and radicicol which bind at the N-terminus (Marcu et al., 2000b). However, this still resulted in inhibition of HSP90 function and degradation of a number of HSP90-chaperoned signalling proteins (Marcu et al., 2000a). Geldanamcyin cannot bind HSP90 subsequent to novobiocin; this suggests that some interaction between the N and C terminal domains must exist and is consistent with the view that both sites are important for HSP90 chaperone properties.

A purine-based HSP90 inhibitor, PU3, has been shown to result in the degradation of signalling molecules, including ERBB2, and to cause cell cycle arrest and differentiation in breast cancer cells (Chiosis et al., 2001).

### HSP90 as a Therapeutic Target

Due to its involvement in regulating a number of signalling pathways that are crucially important in driving the phenotype of a tumour, and the discovery that certain bioactive natural products exert their effects via HSP90 activity, the molecular chaperone HSP90 is currently being assessed as a new target for anticancer drug development (Neckers et al., 1999).

The predominant mechanism of action of geldanamycin, 17AAG, and radicicol involves binding to HSP90 at the ATP binding site located in the N-terminal domain of the protein, leading to inhibition of the intrinsic ATPase activity of HSP90 (see, e.g., Prodromou et al., 1997; Stebbins et al., 1997; Panaretou et al., 1998).

Inhibition of HSP90 ATPase activity prevents recruitment of co-chaperones and encourages the formation of a type of HSP90 heterocomplex from which these client proteins are targeted for degradation via the ubiquitin proteasome pathway (see, e.g., Neckers et al., 1999; Kelland et al., 1999).

Treatment with HSP90 inhibitors leads to selective degradation of important proteins involved in cell proliferation, cell cycle regulation and apoptosis, processes which are fundamentally important in cancer.

Inhibition of HSP90 function has been shown to cause selective degradation of important signalling proteins involved in cell proliferation, cell cycle regulation and apoptosis, processes which are fundamentally important and which are commonly deregulated in cancer (see, e.g., Hostein et al., 2001). An attractive rationale for developing drugs against this target for use in the clinic is that by simultaneously depleting proteins associated with the transformed phenotype, one may obtain a strong antitumour effect and achieve a therapeutic advantage against cancer versus normal cells. These events downstream of HSP90 inhibition are believed to be responsible for the antitumour activity of HSP90 inhibitors in cell culture and animal models (see, e.g., Schulte et al., 1998; Kelland et al., 1999).

### BRIEF DESCRIPTION OF THE INVENTION

The present invention relates to the use of a class of substituted thieno[2,3-d]pyrimidine compounds (referred to herein as pyrimidothiophenes) as HSP90 inhibitors, for example for inhibition of cancer cell proliferation. A core pyrimidothiophene ring with aromatic substitution on one ring carbon atom are principle characterising features of the compounds with which the invention is concerned.

### DETAILED DESCRIPTION OF THE INVENTION

The present invention provides a compound of formula (I), or a salt, N-oxide, hydrate, or solvate thereof:

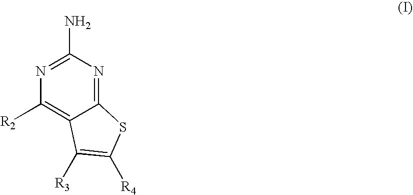

wherein
R₂ is a group of formula (IA):

-(Ar¹)ₘ-(Alk¹)ₚ-(Z)ᵣ-(Alk²)ₛ-Q (IA)
wherein in any compatible combination
Ar¹ is an optionally substituted aryl or heteroaryl radical,
Alk¹ and Alk² are optionally substituted divalent C₁-C₃ alkylene or C₂-C₃alkenylene radicals,
m, p, r and s are independently 0 or 1,
Z is —O—, —S—, —(C═O)—, —(C═S)—, —SO₂—, —C(═O)O—, —C(═O)NR^{A}—, —C(═S)NR^{A}—, —SO₂NR^{A}—, —NR^{A}C(═O)—, —NR^{A}SO₂— or —NR^{A}— wherein R^{A} is hydrogen or C₁-C₆ alkyl, and
Q is hydrogen or an optionally substituted carbocyclic or heterocyclic radical;
   R₃ is hydrogen, an optional substituent, or an optionally substituted (C₁-C₆)alkyl, aryl or heteroaryl radical; and

### R₄ is

(i) hydrogen, a —CN group, a nitro group —NO₂, or a —C(═NOH)(NH₂) group, or
(ii) an optionally substituted C₁-C₆alkyl, aryl, heterocyclic, aryl(C₁-C₆alkyl)-, or heterocyclic(C₁-C₆alkyl)- group, or
(iii) a group of formula —C(═O)R₅ wherein R₅ is hydroxyl, optionally substituted C₁-C₆alkyl, C₁-C₆alkyoxy, aryl, aryloxy, heteroaryl, heteroaryloxy, aryl(C₁-C₆alkyl)-, aryl(C₁-C₆alkoxy)-, heteroaryl(C₁-C₆alkyl)-, or heteroaryl(C₁-C₆alkoxy)-, or
(iv) a group of formula —C(═O)NHR₆ wherein R₆ is primary, secondary, tertiary or cyclic amino, or hydroxyl, optionally substituted C₁-C₆alkyl, C₁-C₆alkyoxy, aryl, aryloxy, heteroaryl, heteroaryloxy, aryl(C₁-C₆alkyl)-, aryl(C₁-C₆alkoxy)-, heteroaryl(C₁-C₆alkyl)-, or heteroaryl(C₁-C₆alkoxy)-.

In one subset of the compounds of the invention, R₄ is a —CN group, or an optionally substituted C₁-C₆alkyl, aryl, heteroaryl, aryl(C₁-C₆alkyl)-, or heteroaryl(C₁-C₆alkyl)- group.

As used herein, the term “(C₁-C₆)alkyl” refers to a straight or branched chain alkyl radical having from 1 to 6 carbon atoms, including for example, methyl, ethyl, n-propyl, isopropyl, n-butyl, isobutyl, sec-butyl, t-butyl, n-pentyl and n-hexyl.

As used herein the term “divalent (C₁-C₆)alkylene radical” refers to a saturated hydrocarbon chain having from 1 to 6 carbon atoms and two unsatisfied valences.

As used herein, the term “(C₁-C₆)alkenyl” refers to a straight or branched chain alkenyl radical having from 2 to 6 carbon atoms and containing at least one double bond of E or Z configuration, including for example, ethenyl and allyl.

As used herein the term “divalent (C₂-C₆)alkenylene radical” refers to a hydrocarbon chain having from 2 to 6 carbon atoms, at least one double bond, and two unsatisfied valences.

As used herein the term “cycloalkyl” refers to a saturated carbocyclic radical having from 3-8 carbon atoms and includes, for example, cyclopropyl, cyclobutyl, cyclopentyl, cyclohexyl, cycloheptyl and cyclooctyl.

As used herein the term “cycloalkenyl” refers to a carbocyclic radical having from 3-8 carbon atoms containing at least one double bond, and includes, for example, cyclopentenyl, cyclohexenyl, cycloheptenyl and cyclooctenyl.

As used herein the term “aryl” refers to a mono-, bi- or tri-cyclic carbocyclic aromatic radical. Illustrative of such radicals are phenyl, biphenyl and napthyl.

As used herein the term “carbocyclic” refers to a cyclic radical whose ring atoms are all carbon, and includes monocyclic aryl, cycloalkyl, and cycloalkenyl radicals.

As used herein the term “heteroaryl” refers to a mono-, bi- or tri-cyclic aromatic radical containing one or more heteroatoms selected from S, N and O. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyrazinyl, triazinyl, indolyl and indazolyl.

As used herein the unqualified term “heterocyclyl” or “heterocyclic” includes “heteroaryl” as defined above, and in particular refers to a mono-, bi- or tri-cyclic non-aromatic radical containing one or more heteroatoms selected from S, N and O, and to groups consisting of a monocyclic non-aromatic radical containing one or more such heteroatoms which is covalently linked to another such radical or to a monocyclic carbocyclic radical. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzfuranyl, pyranyl, isoxazolyl, benzimidazolyl, methylenedioxyphenyl, ethylenedioxyphenyl, maleimido and succinimido groups.

Unless otherwise specified in the context in which it occurs, the term “substituted” as applied to any moiety herein means substituted with at least one substituent, for example selected from (C₁-C₆)alkyl, (C₁-C₆)alkoxy, hydroxy, hydroxy(C₁-C₆)alkyl, mercapto, mercapto(C₁-C₆)alkyl, (C₁-C₆)alkylthio, halo (including fluoro and chloro), trifluoromethyl, trifluoromethoxy, nitro, nitrile (—CN), oxo, phenyl, phenoxy, benzyl, benzyloxy, —COOH, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —NH₂, —NHR^{A}, —NR^{A}R^{B}, —OCONH₂, —OCONHR^{A}, —OCONR^{A}R^{B}, —NHCOR^{A}, —NHCOOR^{A}, —NR^{B}COOR^{A}, —NHSO₂OR^{A}, —NR^{B}SO₂OR^{A}, —NHCONH₂, —NR^{A}CONH₂, —NHCONHR^{B}, —NR^{A}CONHR^{B}, —NHCONR^{A}R^{B} or —NR^{A}CONR^{A}R^{B} wherein R^{A} and R^{B} are independently a (C₁-C₆)alkyl group. An “optional substituent” may be one of the foregoing substituent groups.

As used herein the term “salt” includes base addition, acid addition and quaternary salts. Compounds of the invention which are acidic can form salts, including pharmaceutically or veterinarily acceptable salts, with bases such as alkali metal hydroxides, e.g. sodium and potassium hydroxides; alkaline earth metal hydroxides e.g. calcium, barium and magnesium hydroxides; with organic bases e.g. N-ethyl piperidine, dibenzylamine and the like. Those compounds (I) which are basic can form salts, including pharmaceutically or veterinarily acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic and p-toluene sulphonic acids and the like.

Some compounds of the invention contain one or more actual or potential chiral centres because of the presence of asymmetric carbon atoms. The presence of several asymmetric carbon atoms gives rise to a number of diastereoisomers with R or S stereochemistry at each chiral centre. The invention includes all such diastereoisomers and mixtures thereof.

The radical R₂

As stated, R₂ is a group of formula (IA):

-(Ar¹)ₘ-(Alk¹)ₚ-(Z)ᵣ-(Alk²)ₛ-Q (IA)

wherein in any compatible combination Ar¹ is an optionally substituted aryl or heteroaryl radical, Alk¹ and Alk² are optionally substituted divalent C₁-C₃ alkylene or C₂-C₃ alkenylene radicals, m, p, r and s are independently 0 or 1, Z is —O—, —S—, —(C═O)—, —(C═S)—, —SO₂—, —C(═O)O—, —C(═O)NR^{A}—, —C(═S)NR^{A}—,
—SO₂NR^{A}—, —NR^{A}C(═O)—, —NR^{A}SO₂— or —NR^{A}— wherein R^{A} is hydrogen or C₁-C₆ alkyl, and Q is hydrogen or an optionally substituted carbocyclic or heterocyclic radical

When present in the radical R₂,
Ar¹ may be, for example, a phenyl, cyclohexyl, pyridyl, morpholino, piperidinyl or piperazinyl ring. Presently it is preferred that Ar¹, when present, by a phenyl ring;
Alk¹ and Alk² may be, for example, optionally substituted divalent radicals selected from —CH₂, CH₂CH₂— or —CH═CH—. Optional substituents in Alk¹ and Alk² include, for example mono- or di(C₁-C₃alkyl)amino and C₁-C₃alkoxy; and
Z may be, for example, —O— or —NH—; and Q is hydrogen.

In a simple subclass of compounds with which the invention is concerned, m is 1 and each of p, r and s is 0, and Q is hydrogen, so that R₂ is optionally substituted aryl or heteroaryl. In such cases, R₂ may be, for example, optionally substituted phenyl, 2- or 3-thienyl, 2- or 3-furanyl, 2-, 3- or 4-pyridinyl, morpholinyl, or piperidinyl. Currently preferred are compounds wherein R₂ is optionally substituted phenyl, for example where the optional substituents are selected from methyl, ethyl, n- or isopropyl, vinyl, allyl, methoxy, ethoxy, n-propyloxy, benzyloxy, allyloxy, cyanomethoxy chloro, bromo, cyano, formyl, methyl-, ethyl-, or n-propyl-carbonyloxy, methyl- or ethylaminocarbonyl. More complex substituent groups which may be present in the R₂ ring include those (i) of formula —O(CH₂)ₙZ¹ wherein n is 1, 2 or 3 and Z¹ is a primary, secondary, tertiary or cyclic amino group, or a C₁-C₆alkoxy group; or (ii) of formula -(Alk³)ₘZ¹ wherein Alk³ is a divalent straight or branched chain (C₁-C₃) alkylene, m is 0 or 1, and Z¹ is a primary, secondary, tertiary or cyclic amino group, or a C₁-C₆alkoxy group. Preferred substitution positions in the phenyl ring are positions 2, 4 and 5.

In other simple structures, m is 1, p, r and s are again each 0, and Q may be an optionally substituted carbocyclic or heterocyclic ring, for example phenyl, cyclohexyl, pyridyl, morpholino, piperidinyl, or piperazinyl ring. In such cases, Q is a direct substituent in the optionally substituted Ar¹ ring.

In more complex structures with which the invention is concerned, one or more of m, p, r and s may be 1, and Q may be hydrogen or an optionally substituted carbocyclic or heterocyclic ring. For example, p and/or s may be 1 and r may be 0, so that Q is linked to Ar¹ by an alkylene or alkenylene radical, for example a C₁-C₃ alkylene radical, which is optionally substituted. In other cases each of p, r, and s may be 1, in which cases, Q is linked to Ar¹ by an alkylene or alkenylene radical which is interrupted by the hetero atom-containing Z radical. In still other cases, p and s may be 0 and r may be 1, in which case Q is linked to Ar¹ via the hetero atom-containing Z radical.

Specific examples of R₂ groups usable in compounds of the invention include those present in the compounds of the Examples herein.

### The Optional Substituent R₃

R₃ is hydrogen or an optional substituent, as defined above. Presently it is preferred that R₃ be hydrogen.

### The Group R₄

In one specific subclass of compounds of the invention, R₄ is hydrogen, nitrile, or —C(═NOH)(NH₂)

In another subclass, R₄ is an imidazolyl or oxadiazolyl group, a C₁-C₆alkyl group, optionally substituted by a hydroxyl or primary, secondary, tertiary or cyclic amino group, or a group of formula —C(═O)R₅ wherein R₅ is C₁-C₆alkyl or phenyl, or a group of formula —C(═O)NHR₆ wherein R₆ is N— piperidinyl, N-morpholinyl, N-piperazinyl, N¹-methyl-N-piperazinyl, N-triazolyl, C₁-C₆alkyoxy, or mono or di-C₁-C₆alkylamino.

In one preferred subclass of compounds of the invention R₄ is an optionally substituted phenyl, phenyl(C₁-C₆alkyl)-, heterocyclic or heterocyclic(C₁-C₆alkyl)- group wherein the heterocyclic part is monocyclic with 5 or 6 ring atoms. This subclass includes compounds wherein R₄ is an optionally oxadiazolyl, imidazolyl, dihydro-imidazolyl, triazolyl, pyrazolyl, pyrrolyl, thiazolyl or tetrazolyl group. Specifically, in this subclass, R₄ may be an oxadiazol-3-yl, 4,5-dihydro-1H-imidazol-2-yl, [1,2,4]triazol-4-yl, 5-amino-1H-[1,2,4]triazol-3-yl, 4- or 5-methyl-2H-pyrazol-3-yl, 1H-pyrrol-2-yl, 2-amino-5-methyl-thiazol-4-yl, 3H-imidazol-4-yl, or 2H-tetrazol-5-yl group.

In another preferred subclass of compounds of the invention, R₄ is optionally substituted methyl, ethyl or n-propyl. In this subclass, substituents in R₄ may be selected from amino, methylamino, ethylamino, n-propylamino, acetamido, oxo, hydroxyl, phenyl, methyl, ethyl, and n-propyl. Specifically, in this subclass, R₄ may be acetamidomethyl, formyl, 2-hydroxy-2-methyl-propyl, 2-hydroxy-2-ethyl-but-1-yl, hydroxymethyl, ethylcarbonyl, phenylcarbonyl, n-propylaminomethyl, aminomethyl, or diphenyl-hydroxymethyl,

Specific examples of R₄ groups usable in compounds of the invention include those present in the compounds of the Examples herein.

A preferred subclass of the compounds with which the invention is concerned has formula (II):

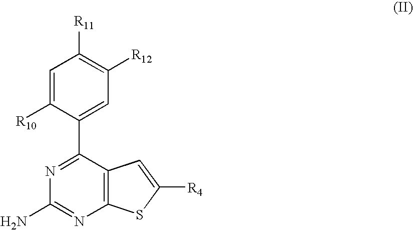

wherein
R₄ is as defined and discussed above;

### R₁₀ is H, Cl, Br, or CH₃;

R₁₁ is hydrogen, Cl, Br, CN, methyl, ethyl, n- or iso-propyl, vinyl or allyl; R₁₂ is (i) a radical of formula —O(CH₂)ₙZ¹ wherein n is 1, 2 or 3 and Z¹ is a primary, secondary, tertiary or cyclic amino group, or a C₁-C₆alkoxy group; or (ii) a radical of formula -(Alk³)ₘZ¹ wherein Alk³ is a divalent straight or branched chain (C₁-C₃) alkylene, m is 0 or 1, and Z¹ is a primary, secondary, tertiary or cyclic amino group, or a C₁-C₆alkoxy group.

Specific compounds with which the invention is concerned include those of the Examples.

Compounds with which the invention is concerned may be prepared by literature methods, such as those of the preparative Examples herein, and methods analogous thereto. For example the following general reaction scheme can be employed:

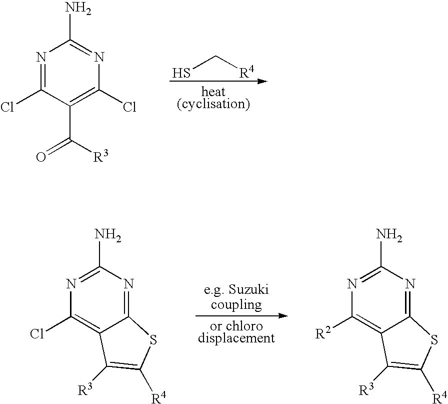

Starting material are either available commercially or can be made according to literature methods. Subsequent reactions may be carried out on R², R³ or R⁴ to prepare additional compounds of formula (I)

The compounds of the invention are inhibitors of HSP90 and are useful in the treatment of diseases which are responsive to inhibition of HSP90 activity such as cancers; viral diseases such as Hepatitis C(HCV) (Waxman, 2002); Immunosupression such as in transplantation (Bijlmakers, 2000 and Yorgin, 2000); Anti-inflammatory diseases (Bucci, 2000) such as Rheumatoid arthritis, Asthma, MS, Type I Diabetes, Lupus, Psoriasis and Inflammatory Bowel Disease; Cystic fibrosis (Fuller, 2000); Angiogenesis-related diseases (Hur, 2002 and Kurebayashi, 2001): diabetic retinopathy, haemangiomas, psoriasis, endometriosis and tumour angiogenesis. Also an Hsp90 inhibitor of the invention may protect normal cells against chemotherapy-induced toxicity and be useful in diseases where failure to undergo apoptosis is an underlying factor. Such an Hsp90 inhibitor may also be useful in diseases where the induction of a cell stress or heat shock protein response could be beneficial, for example, protection from hypoxia-ischemic injury due to elevation of Hsp70 in the heart (Hutter, 1996 and Trost, 1998) and brain (Plumier, 1997 and Rajder, 2000). An Hsp90 inhibitor-induced increase in Hsp70 levels could also be useful in diseases where protein misfolding or aggregation is a major causal factor, for example, neurogenerative disorders such as scrapie/CJD, Huntingdon's and Alzheimer's (Sittler, 2001; Trazelt, 1995 and Winklhofer, 2001)”.

Accordingly, the invention also includes:

(i) A pharmaceutical or veterinary composition comprising a compound of formula (I) above, together with a pharmaceutically or veterinarily acceptable carrier.
(ii) The use of a compound a compound of formula (I) above in the preparation of a composition for composition for inhibition of HSP90 activity in vitro or in vivo.
(iii). A method of treatment of diseases or conditions which are responsive to inhibition of HSP90 activity in mammals which method comprises administering to the mammal an amount of a compound of formula (I) above effective to inhibit said HSP90 activity.

It will be understood that the specific dose level for any particular patient will depend upon a variety of factors including the activity of the specific compound employed, the age, body weight, general health, sex, diet, time of administration, route of administration, rate of excretion, drug combination and the causative mechanism and severity of the particular disease undergoing therapy. In general, a suitable dose for orally administrable formulations will usually be in the range of 0.1 to 3000 mg, once, twice or three times per day, or the equivalent daily amount administered by infusion or other routes. However, optimum dose levels and frequency of dosing will be determined by clinical trials as is conventional in the art.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

The following examples illustrate the preparation and activities of specific compounds of the invention:

### General Procedures

All reagents obtained from commercial sources were used without further purification. Anhydrous solvents were obtained from commercial sources and used without further drying. Flash chromatography was performed with pre-packed silica gel cartridges (Strata SI-1; 61 Å, Phenomenex, Cheshire UK or IST Flash II, 54 Å, Argonaut, Hengoed, UK). Thin layer chromatography was conducted with 5×10 cm plates coated with Merck Type 60 F₂₅₄ silica gel.

The compounds of the present invention were characterized by LC/MS using a Hewlett Packard 1100 series LC/MSD linked to quadrupole detector (ionization mode: electron spray positive); column: Phenomenex Luna 3u C18(2) 30×4.6 mm; Buffer A prepared by dissolving 1.93 g ammonium acetate in 2.5 L HPLC grade H₂O and adding 2 mL formic acid. Buffer B prepared by adding 132 mL buffer A to 2.5 L of HPLC grade acetonitrile and adding 2 mL formic acid; elution gradient 95:5 to 5:95 buffer A: buffer B over 3.75 minutes. Flow rate=2.0 mL/min). In some instances the same gradient and flow rate were run over a period of seven minutes. Run times are specified for each example.

Preparative HPLC Purification (standard method) was carried out using a Waters preparative HPLC with fraction collection mass directed. The mass detector is a micromass ZQ series 2000, ionisation mode: electron spray positive, column: Phenomenex Gemini C18 5□nm, 100×21.2 mm. Buffer A: 0.08% (v/v) formic acid, 20 mM ammonium acetate. Buffer B: 0.08% (v/v) formic acid, 5% (v/v) A. Flow rate 20 ml/min.

Nuclear magnetic resonance (NMR) analysis was performed with a Brucker DPX-400 MHz NMR spectrometer. The spectral reference was the known chemical shift of the solvent. Proton NMR data is reported as follows: chemical shift (δ) in ppm, multiplicity (s=singlet, d=doublet, t=triplet, q=quartet, p=pentet, m=multiplet, dd=doublet of doublet, br=broad), coupling constant, integration.

### EXAMPLE 1

### 2-Amino-4-(2,4-dichloro-phenyl)-thieno[2,3-d]pyrimidine-6-carboxylic acid

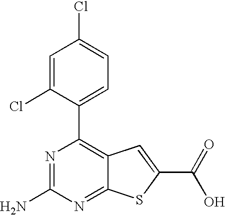

### Step 1

### 2-Amino-4-chloro-thieno[2,3-d]pyrimidine-6-carboxylic acid ethyl ester

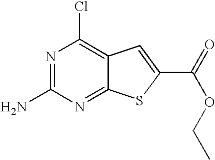

To a stirred mixture of 2-amino-4,6-dichloro-5-formyl-pyrimidine (1 eq.) and potassium carbonate (1 eq.) in acetonitrile at ambient temperature was added ethyl-2-mercaptoacetate (0.95 eq.) and the mixture stirred at ambient temperature for three hours, followed by heating at 80° C. for one hour. After cooling, the mixture was concentrated to dryness in vacuo. Column chromatography on silica gel, eluting with ethyl acetate and hexanes, gave the product as a yellow powder.

LC-MS retention time: 2.371 minutes, [M+H]⁺ 258

### Step 2 (Suzuki Reaction):

### 2-Amino-4-(2,4-dichloro-phenyl-thieno[2,3-d]pyrimidine-6-carboxylic acid ethyl ester

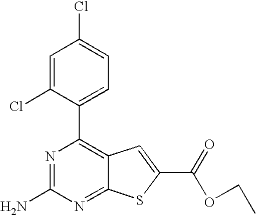

Dimethylformamide (50 ml) was added to a mixture of 2-Amino-4-chloro-thieno[2,3-d]pyrimidine-6-carboxylic acid ethyl ester (2.86 g; 0.0111 mole), 2,4-dichlorophenylboronic acid (2.78 g; 0.0144 mole) and sodium hydrogen carbonate (2.79 g; 0.0333 mole). Water (10 ml) was then added and the resulting suspension was degassed by evacuation-nitrogen purge; then bubbling of nitrogen gas through the reaction mixture for 5 minutes. Dichloro-bis-triphenylphosphine palladium (II) (388 mg; 5 mole %) was added and the reaction mixture was heated at 85° C. for 5.5 hours. Reaction mixture was allowed to cool and solvents were removed in vacuo. The residue was partitioned between ethyl acetate (350 ml) and water (200 ml). Mixture was stirred vigorously for 10 minutes then filtered through a pad of celite to remove Pd residual solids. Filtrate phases were separated and the organic phase was washed with water (200 ml) then saturated aqueous sodium chloride solution (200 ml). The organic phase was dried over Na₂SO₄, filtered and filtrate solvents removed in vacuo to afford a brown solid. Product was triturated with ethyl acetate-hexane mixture to afford product as a yellow solid (2.291 g; 56%)

LC-MS retention time: 2.741 minutes, [M+H]⁺ 370, 368

### Step 3 (Ester Hydrolysis)

### 2-Amino-4-(2,4-dichloro-phenyl)-thieno[2,3-d]pyrimidine-6-carboxylic acid

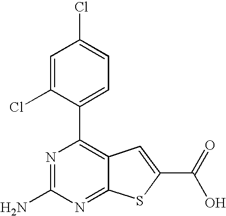

Sodium hydroxide (0.988 g; 0.0248 mole) was added to a suspension of 2-amino-4-(2,4-dichloro-phenyl-thieno[2,3-d]pyrimidine-6-carboxylic acid ethyl ester (4.548 g; 0.0124 mole) in a mixture of ethanol (100 ml) and water (10 ml). The reaction mixture was heated to reflux for 1 hour 15 minutes then allowed to cool to ambient temperature. Solvents were removed in vacuo and residue was suspended in water (100 ml). The mixture was neutralised by drop-wise addition of hydrochloric acid solution (2.0M), then freeze-dried to afford product as light brown solid (containing 2 equivalents of sodium chloride), 5.626 g; 99%. A small sample was further purified by preparative HPLC.

LC-MS retention time: 2.185 minutes, [M+H]⁺ 342, 340

This compound had activity ‘A’ in the fluorescence polarization assay described below.

### EXAMPLE 2

### 2-Amino-4-(2,4-dimethyl-phenyl)-thieno[2,3-d]pyrimidine-6-carboxylic acid

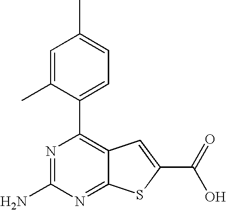

Prepared as for example 1 using 2,4 dimethyl phenyl boronic acid in the Suzuki coupling reaction (step 2).

LC-MS retention time: 1.959 minutes, [M+H]⁺ 300

This compound had activity ‘B’ in the fluorescence polarization assay described below.

### EXAMPLE 3

### 2-[2-Amino-4-(2,4-dichloro-phenyl)-thieno[2,3-d]pyrimidin-6-yl]-propan-2-ol

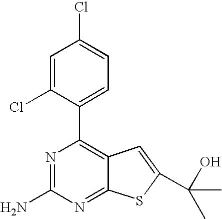

2-Amino-4-(2,4-dichloro-phenyl)-thieno[2,3-d]pyrimidine-6-carboxylic acid ethyl ester (200 mg, 0.543 mmol, 1 eq) was dissolved in anhydrous tetrahydrofuran (5 ml) and cooled to −78° C. under a nitrogen atmosphere. Methyl Magnesium Bromide (3M in diethyl ether, 0.543 ml, 3 eq) was added, and the reaction warmed to room temperature and stirred for 20 hours. The mixture was quenched with water, diluted to 100 ml with water and pH adjusted to pH1 by addition of aqueous hydrochloric acid (2.0M). The mixture was extracted with dichloromethane (2×100 ml), and the combined organic extracts were washed with saturated brine (50 ml) and dried over Na₂SO₄. The solvent was evaporated and the brown residue was purified on silica gel (eluting with 1:50 methanol:dichloromethane). The resulting yellow residue was triturated with diethyl ether. Yield: 38 mg (20%)

LC-MS retention time: 2.369 minutes, [M+H]⁺ 354 and 356 with 2 Cl splitting pattern This compound had activity ‘B’ in the fluorescence polarization assay described below.

### EXAMPLE 4

### 3-[2-Amino-4-(2,4-dichloro-phenyl)-thieno[2,3-d]pyrimidin-6-yl]-pentan-3-ol

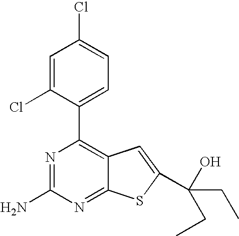

Prepared as for example 3.

LC-MS retention time: 2.614 minutes, [M+H]⁺ 382 and 384 with 2 Cl splitting pattern.

This compound had activity ‘A’ in the fluorescence polarization assay described below.

### EXAMPLE 5

### [2-Amino-4-(2,4-dichloro-phenyl)-thieno[2,3-d]pyrimidin-6-yl]-diphenyl-methanol

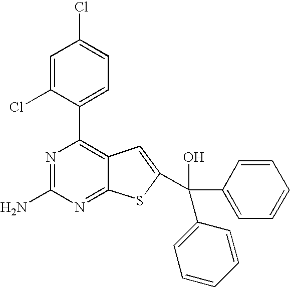

Prepared as for example 3.

LC-MS retention time: 2.791 minutes, [M+H]⁺ 478 and 480 with 2 Cl splitting pattern.

This compound had activity ‘B’ in the fluorescence polarization assay described below.

### EXAMPLE 6

### [2-Amino-4-(2,4-dichloro-phenyl)-thieno[2,3-d]pyrimidin-6-yl]methanol

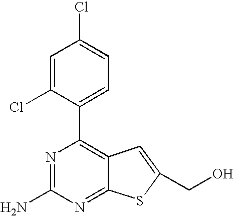

2-Amino-4-(2,4-dichloro-phenyl)-thieno[2,3-d]pyrimidine-6-carboxylic acid ethyl ester (0.50 g, 1.36 mmol, 1 eq) was dissolved in anhydrous THF (8 ml) at −78° C. under a nitrogen atmosphere. Diisobutyl aluminium hydride (1M in THF, 5.43 ml, 4 eq) was added. The reaction was warmed to room temperature and then stirred for 30 minutes. The black reaction mixture was poured onto crushed ice, acidified with 10 ml 2M aqueous HCl and extracted into 500 ml dichloromethane. The organic phase was washed with saturated brine and dried over Na₂SO₄ and evaporated in vacuo. The light brown residual solid was purified on silica gel eluting with a mixture of ethyl acetate and hexane. The resulting light brown solid was triturated with diethyl ether to yield an off-white solid (0.185 g, 42%).

LC-MS retention time: 2.185 minutes, [M+H]⁺ 326 and 328 with 2 Cl splitting pattern.

This compound had activity ‘A’ in the fluorescence polarization assay described below.

### EXAMPLE 7

### 2-Amino-4-(2,4-dimethylphenyl)-thieno[2,3-d]pyrimidine-6-carbonitrile

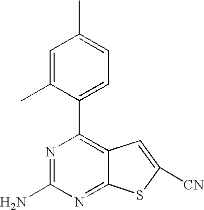

### Step 1

### 2-Amino-4-(2,4-dimethylphenyl)-thieno[2,3-d]pyrimidine-6-carboxylic acid ethyl ester

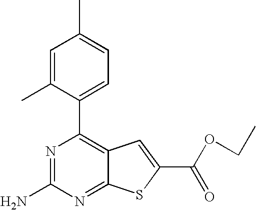

Prepared as for example 1 (step 2)

### Step 2

### 2-Amino-4-(2,4-dimethylphenyl)-thieno[2,3-d]pyrimidine-6-carboxylic acid amide

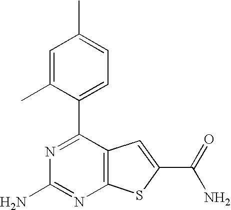

A suspension of 2-Amino-4-(2,4-dimethylphenyl)-thieno[2,3-d]pyrimidine-6-carboxylic acid ethyl ester in methanolic ammonia (7N) was heated in a sealed tube at 85° C., for 72 hrs. The resulting solution was concentrated and the residue triturated with diethyl ether to give a pale yellow powder.

### Step 3

### 2-Amino-4-(2,4-dimethylphenyl)-thieno[2,3-d]pyrimidine-6-carbonitrile

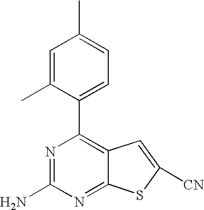

Trifluoroacetic anhydride was added to a solution of 2-Amino-4-(2,4-dimethylphenyl)-thieno[2,3-d]pyrimidine-6-carboxylic acid amide in pyridine/dichloromethane, at ˜0° C. (ice/water bath), and the solution stirred for ˜2 hrs. Water was added and the mixture washed with aqueous ammonia (0.880), water and saturated aqueous sodium chloride solution. Solution was concentrated and the residue triturated with diethyl ether, to give a pale yellow solid.

LC retention time 2.597 minutes [M+H]⁺ 281 (Run time 3.75 mins)

This compound had activity ‘A’ in the fluorescence polarization assay described below.

### EXAMPLE 8

### 1-[2-Amino-4-(2,4-dichloro-phenyl)-thieno[2,3-d]pyrimidine-6-yl]-propan-1-one

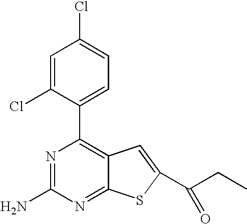

### Step 1

### 2-Amino-4-(2,4-dichloro-phenyl)-thieno[2,3-d]pyrimidine-6-carbaldehyde

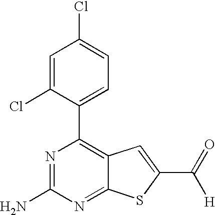

[2-Amino-4-(2,4-dichloro-phenyl)-thieno[2,3-d]pyrimidin-6-yl]methanol (1.77 g, 5.427 mmol, 1 eq) was dissolved in acetone (150 ml). Jones reagent (2.59M, 2.096 ml, 5.427 mmol, 1 eq) was added upon which a green precipitate formed. The reaction mixture was stirred at room temperature for 1 hour, quenched with methanol and filtered through celite. The clear solution was evaporated to dryness and the yellow residue partitioned between ethyl acetate (800 ml) and saturated sodium hydrogen carbonate solution (2×300 ml). The organic layer was washed with water (300 ml) and saturated brine (300 ml) and evaporated to yield a yellow solid (1.22 g, 69%).

LC-MS retention time: 2.489 minutes, [M+H]⁺ 324 and 326 with 2 Cl splitting pattern.

### Step 2

### 1-[2-Amino-4-(2,4-dichloro-phenyl)-thieno[2,3-d]pyrimidin-6-yl]-propan-1-ol

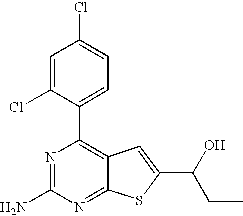

To a solution of 2-Amino-4-(2,4-dichloro-phenyl)-thieno[2,3-d]pyrimidine-6-carbaldehyde (150 mg, 0.463 mmol, 1 eq) in anhydrous THF (5 ml) under a nitrogen atmosphere was added ethyl magnesium bromide (1M in THF, 2.31 ml, 5 eq at) −78° C. The reaction was warmed to room temperature and then stirred at reflux for 4 hours, after which another 2.31 ml (5 eq) of ethyl magnesium bromide were added, followed by 4 hours heating at reflux. was hence quenched with water, acidified with dilute HCl and extracted into ethyl acetate. The organic layer was evaporated to dryness and purified on a 5 g silica gel cartridge. Unreacted aldehyde eluted with 1:5 ethyl acetate:hexane, the desired product with 1:1 ethyl acetate:hexane. Yield: 15 mg (9%).

LC-MS retention time: 2.418 minutes, [M+H]⁺ 354 and 356 with 2 Cl splitting pattern.

### Step 3

### 1-[2-Amino-4-(2,4-dichloro-phenyl)-thieno[2,3-d]pyrimidin-6-yl]-propan-1-one

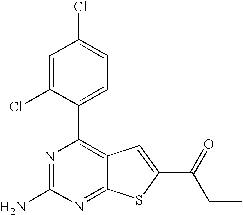

1-[2-Amino-4-(2,4-dichloro-phenyl)-thieno[2,3-d]pyrimidin-6-yl]-propan-1-ol (15 mg, 0.042 mmol, 1 eq) was dissolved in acetone (4 ml) and Jones reagent (2.59M, 16.35 μL, 1 eq) was added. After stirring for 30 minutes, the green precipitate was filtered off through a pad of celite and the filtrate evaporated to dryness. The sample was purified by preparative HPLC. Yield: 9.4 mg (64%).

LC-MS retention time: 2.632 minutes, [M+H]⁺ 352 and 354 with 2 Cl splitting pattern.

This compound had activity ‘A’ in the fluorescence polarization assay described below.

### EXAMPLE 9

### [2-Amino-4-(2,4-dichloro-phenyl)-thieno[2,3-d]pyrimidine-6-yl]-phenyl-methanone

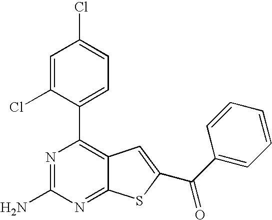

Prepared as for example 8

LC-MS retention time: 2.771 minutes, [M+H]⁺ 400 and 402 with 2 Cl splitting pattern.

This compound had activity ‘A’ in the fluorescence polarization assay described below.

### EXAMPLE 10

### 4-(2,4-dimethyl-phenyl)-6-[1,2,4]oxadiazol-3-yl-thieno[2,3-d]pyrimidin-2-ylamine

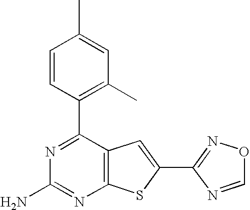

### Step 1

### 2-Amino-4-(2,4-dimethyl-phenyl)-N-hydroxy-thieno[2,3-d]pyrimidine-6-carboxamidine

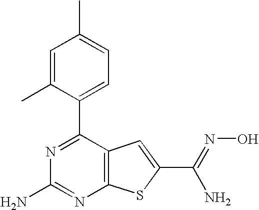

Hydroxylamine hydrochloride and sodium acetate were added to a suspension of 2-Amino-4-(2,4-dimethylphenyl)-thieno[2,3-d]pyrimidine-6-carbonitrile in ethanol and the suspension heated under reflux for 90 minutes. The resulting suspension was allowed to cool and water added to give a pale yellow precipitate. Solids were removed by filtration and washed with water, to give a pale yellow powder which was dried in vacuo.

### Step 2

### 4-(2,4-dimethyl-phenyl)-6-[1,2,4]oxadiazol-3-yl-thieno[2,3-d]pyrimidin-2-ylamine

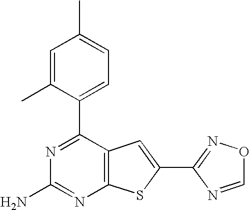

Triethyl orthoformate was added to a solution of 2-Amino-4-(2,4-dimethylphenyl)-thieno[2,3-d]pyrimidine-6-carboxamidine in 1,4-dioxan under a nitrogen atmosphere. Boron trifluoride etherate (cat.) was added and the solution heated, 100° C., for 90 mins. The resulting suspension was allowed to cool and dichloromethane added, the solution was washed with aqueous ammonia (0.880), water, and saturated aqueous sodium chloride solution. The solution was dried over anhydrous sodium sulphate and concentrated to a dark red solid which was purified by chromatography on silica gel eluting with mixtures of ethyl acetate and hexane.

LC retention time 2.554 minutes [M+H]⁺ 324 (Run time 3.75 mins)

This compound had activity ‘A’ in the fluorescence polarization assay described below.

### EXAMPLE 11

### 6-(4,5-Dihydro-1H-imidazol-2-yl)-4-(2,4-dimethyl-phenyl)-thieno[2,3-d]pyrimidin-2-ylamine

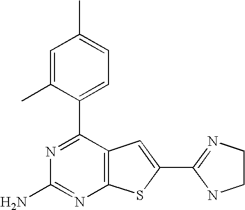

Ethylene diamine was added to a solution of 2-Amino-4-(2,4-dimethylphenyl)-thieno[2,3-d]pyrimidine-6-carboxamidine (example 10 step 1) in acetic acid and the solution heated at 125° C. for 18 hrs. The resulting solution was allowed to cool and concentrated to a pale brown semi-solid. The residue was taken up in dichloromethane and washed with aqueous ammonia (0.880) and saturated aqueous sodium chloride solution. The solution was dried over anhydrous sodium sulphate and concentrated to a pale yellow/green gum. The resulting residue was purified by preparative HPLC.

LC retention time 1.672 minutes [M+H]⁺ 324.2 (Run time 3.75 mins)

This compound had activity ‘A’ in the fluorescence polarization assay described below.

### EXAMPLE 12

### 2-Amino-4-(2,4-dichloro-phenyl)-thieno[2,3-d]pyrimidine-6-carboxylic acid (4-methyl-piperazin-1-yl) amide

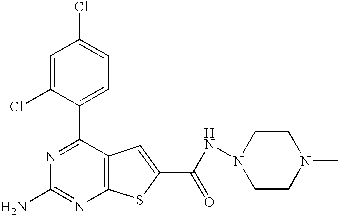

O-(7-Azabenzotriazol-1-yl)-N,N,N′,N′-tetramethyluronium hexafluorophosphate (0.108 g, 0.284 mmol) was added to 2-amino-4-(2,4-dichlorophenyl)-thieno[2,3-d]pyrimidine-6-carboxylic acid (0.100 g, 0.219 mmol) (compound of example 1). This mixture was suspended in dimethylformamide (DMF) (2.5 ml) and diisopropylethylamine (115 μL; 0.657 mmol) added to afford a yellow solution. 1-amino-4-ethylpiperazine (35 μL; 0.284 mmol) was added and the reaction mixture was heated for ten minutes at 100° C. in a sealed vial in a microwave synthesiser. DMF was removed in vacuo and the residue was partitioned between ethyl acetate (20 ml) and water (20 ml). The phases were separated and the organic phase was washed with saturated sodium chloride solution and dried over sodium sulphate. Mixture was filtered and the filtrate solvents were removed in vacuo to leave a yellow solid which was purified by preparative HPLC

LC retention time 1.716 minutes [M+H]⁺ 439, 437 (Run time 3.75 mins)

This compound had activity ‘A’ in the fluorescence polarization assay described below.

### EXAMPLE 13

### 2-Amino-4-(2,4-dichloro-phenyl)-thieno[2,3-d]pyrimidine-6-carboxylic acid [1,2,4]triazol-4-ylamide

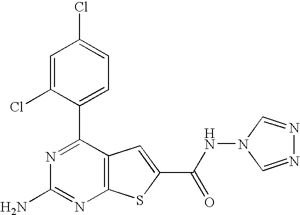

Prepared as for example 12

LC retention time 1.977 minutes [M+H]⁺ 408, 406 (Run time 3.75 mins)

This compound had activity ‘A’ in the fluorescence polarization assay described below.

The following compounds (Table 1) were made by the method of Example 12 from the corresponding carboxylic acid (made by the method of example 1) and the appropriate hydroxylamine or hydrazine derivative.

The final column of Table 1 states the activity of the compound in the fluorescence polarization assay described below.

TABLE 1

| Exam- | | | Hsp90 |
|---|---|---|---|
| ple | Structure | MH+ | FP IC50 |
| 14 | 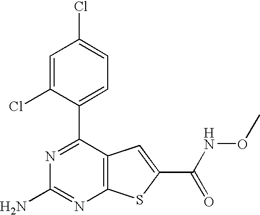 | 370 | A |
| 15 | 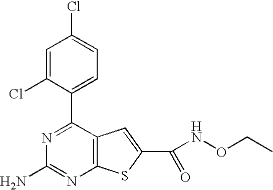 | 384 | A |
| 16 | 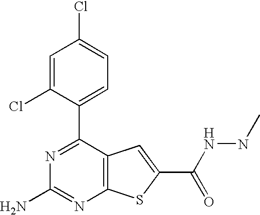 | 369 | A |
| 17 | 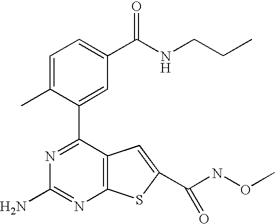 | 400 | A |

### EXAMPLE 18

### 4-(2,4-dimethyl-phenyl)-6-propylaminomethyl-thieno[2,3-d]pyrimidin-2-ylamine

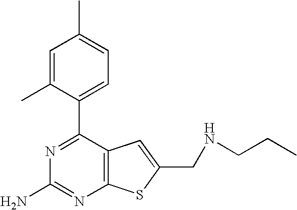

### Step 1 (Amide Synthesis)

### 2-Amino-4-(2,4-dimethyl-phenyl)-thieno[2,3-d]pyrimidine-6-carboxylic acid propylamide

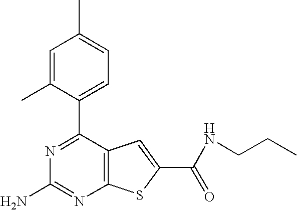

Prepared as for example 12 from the compound of example 2 and n-propylamine.

### Step 2 (Amide Reduction)

### 4-(2,4-dimethyl-phenyl)-6-propylaminomethyl-thieno[2,3-d]pyrimidin-2-ylamine

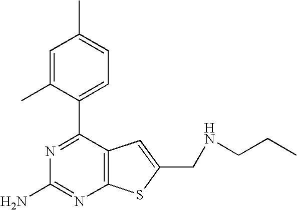

A solution of lithium aluminium hydride (1.0M; 0.300 ml; 0.30 mmol) was added dropwise to a solution of 2-Amino-4-(2,4-dimethyl-phenyl)-thieno[2,3-d]pyrimidine-6-carboxylic acid propylamide (0.050 g; 0.15 mmol) in anhydrous THF (4.0 ml) under a nitrogen atmosphere. The reaction mixture was heated to reflux for two hours then allowed to cool to room temperature. Water was added (0.15 ml) followed by 1.0M sodium hydroxide solution (0.15 ml). The reaction mixture was diluted with ethyl acetate then filtered through a small pad of celite. The filtrate solvents were removed in vacuo to afford a brown oil which was purified by preparative HPLC to afford product as an off-white solid (9 mg; 19%)

LC retention time 1.719 minutes [M+H]⁺ 327 (Run time 3.75 mins)

This compound had activity ‘A’ in the fluorescence polarization assay described below.

### EXAMPLE 19

### 4-(2,4-Dichloro-phenyl)-6-(5-methyl-2H-pyrazol-3-yl)-thieno[2,3-d]pyrimidin-2-ylamine

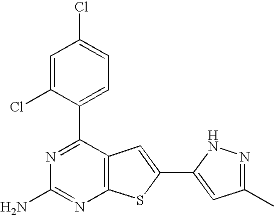

### Step 1

### 4-[2-amino-4-(2,4-dichloro-phenyl)-thieno[2,3-d]pyrimidin-6-yl]-but-3-en-2-one

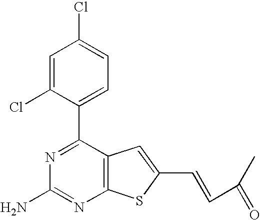

1-(Triphenylphosphoranylidene)-2-propanone was added to a suspension of 2-Amino-4-(2,4-dichloro-phenyl)-thieno[2,3-d]pyrimidine-6-carbaldehyde (example 8 step 1) in toluene and the mixture heated at ˜95° C., for 120 minutes. The resulting suspension was allowed to cool and hexane added. The solids were removed by filtration, washed with hexane and dried in vacuo to give the product as a yellow solid.

LC retention time 2.550 minutes [M+H]⁺ 363.9/365.9 (Run time 3.75 mins)

### Step 2

### 4-(2,4-Dichloro-phenyl)-6-(5-methyl-2H-pyrazol-3-yl)-thieno[2,3-d]pyrimidin-2-ylamine

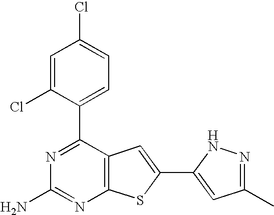

P-toluene sulphonylhydrazide was added to a suspension of 4-[2-Amino-4-(2,4-dichloro-phenyl)-thieno[2,3-d]pyramid-6-yl]-but-3-en-2-one in ethanol and the mixture heated at ˜80° C., for ˜90 minutes The resulting solution was allowed to cool and sodium ethoxide added. The mixture was then heated at ˜80° C., for 4 hrs, then the resulting solution was allowed to cool to ambient temperature and ethyl acetate added. The mixture was washed with saturated ammonium chloride solution, water and saturated aqueous sodium chloride solution. The solution was dried over anhydrous sodium sulphate and concentrated to a yellow solid. The crude product was purified by preparative HPLC, to give the product as a yellow solid.

LC retention time 2.428 minutes [M+H]⁺ 376/377.9 (Run time 3.75 mins)

This compound had activity ‘A’ in the fluorescence polarization assay described below.

### EXAMPLE 20

### 4-(2,4-Dichloro-phenyl)-6-(4-methyl-2H-pyrazol-3-yl)-thieno[2,3-d]pyrimidin-2-ylamine

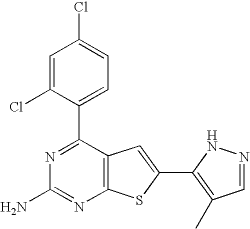

### Step 1

### 4-(2,4-Dichloro-phenyl)-6-(3,3-dimethoxy-2-methyl-propenyl)-thieno[2,3-d]pyrimidin-2-ylamine

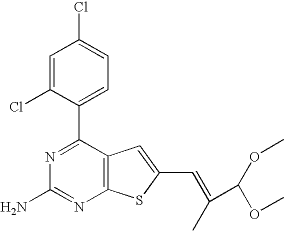

2-(Triphenylphosphoranylidene)-propionaldehyde was added to a suspension of 2-Amino-4-(2,4-dichloro-phenyl)-thieno[2,3-d]pyrimidine-6-carbaldehyde (step 1 example 8) in toluene and the mixture heated at ˜95° C., for ˜120 minutes. The resulting solution was allowed to cool and concentrated to an orange solid. The crude product was purified by ion-exchange chromatography, eluting with mixtures of dichloromethane/methanol and triethyl amine (dimethyl acetal formed during purification). The crude product was re-purified by column chromatography, eluting with mixtures of ethyl acetate and hexane, to give the product as a yellow gum.

LC retention time 2.760 minutes [M+H]⁺ 410/412 (Run time 3.75 mins)

### Step 2

### 4-(2,4-Dichloro-phenyl)-6-(4-methyl-2H-pyrazol-3-yl)-thieno[2,3-d]pyrimidin-2-ylamine

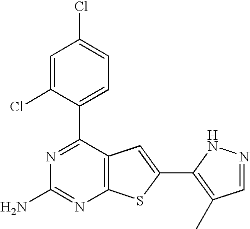

P-toluene sulphonylhydrazide was added to a suspension of 4-(2,4-dichloro-phenyl)-6-(3,3-dimethoxy-2-methyl-propenyl)-thieno[2,3-d]pyramid-2-ylamine in ethanol and the mixture heated at ˜80° C., for ˜90 minutes. The resulting solution was allowed to cool and sodium ethoxide added. The mixture heated at ˜80° C., for ˜2 hrs, the resulting solution was allowed to cool and concentrated. The residue was taken up in ethyl acetate and then washed with saturated ammonium chloride solution, water and saturated aqueous sodium chloride solution. The solution was dried over anhydrous sodium sulphate and concentrated to a yellow solid. The crude product was purified by column chromatography on silica gel, eluting with mixtures of dichloromethane and methanol to give the product as a yellow solid, solids were washed with diethyl ether and dried in vacuo.

LC retention time 2.450 minutes [M+H]⁺ 376/377.9 (Run time 3.75 mins)

This compound had activity ‘A’ in the fluorescence polarization assay described below.

### EXAMPLE 21

### 4-(2,4-Dichloro-phenyl)-6-(1H-pyrrol-2-yl)-thieno[2,3-d]pyrimidin-2-ylamine

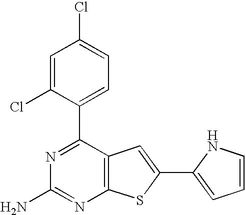

### Step 1

### 1-[2-Amino-4-(2,4-dichloro-phenyl)-thieno[2,3-d]pyrimidin-6-yl]-3-[1,3]dioxan-2-yl-propan-1-ol

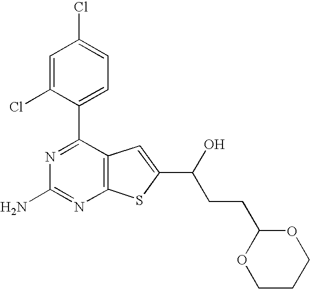

(1,3-Dioxan-2-ylethyl)magnesium bromide was added to a solution of 2-Amino-4-(2,4-dichloro-phenyl)-thieno[2,3-d]pyrimidine-6-carbaldehyde (step 1; example 8) in 1,4-dioxan at ˜−78° C., under a nitrogen atmosphere. The resulting solution was stirred for 60 minutes at ˜−78° C. and for ˜90 minutes at room temperature. The resulting suspension was cooled and saturated aqueous ammonium chloride solution added. The mixture was extracted with ethyl acetate and the extracts washed with water and saturated aqueous sodium chloride solution. The solution was dried over anhydrous sodium sulphate and concentrated to an orange gum. The crude product was purified by column chromatography on silica gel, eluting with mixtures of ethyl acetate and hexane, to give the product as a yellow gum.

LC retention time 2.337 minutes [M+H]⁺ 440/442 (Run time 3.75 mins)

### Step 2

### 1-[2-Amino-4-(2,4-dichloro-phenyl)-thieno[2,3-d]pyrimidin-6-yl]-3-[1,3]dioxin-2-yl-propan-1-one

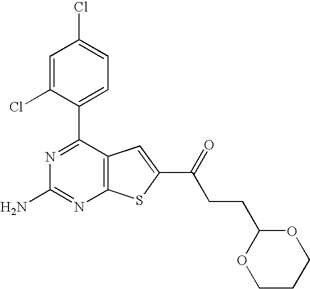

Manganese (IV) oxide (15 equivalents) was added to a solution of 1-[2-Amino-4-(2,4-dichlorophenyl)thieno[2,3-d]pyrimidin-6-yl]-3-[1,3]dioxin-2-yl-propan-1-ol in 1,4-dioxan and the mixture heated at ˜100° C., for 90 minutes. The resulting suspension was filtered and the filtrate concentrated to a yellow-green solid. Solids were washed with hexane and dried in vacuo.

LC retention time 2.583 minutes [M+H]⁺ 438/440 (Run time 3.75 mins)

### Step 3

### 4-(2,4-Dichloro-phenyl)-6-(1H-pyrrol-2-yl)-thieno[2,3-d]pyrimidin-2-ylamine

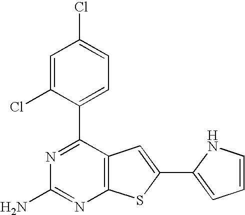

Hydrochloric acid (aq) (˜6M) was added to a solution of 1-[2-Amino-4-(2,4-dichlorophenyl)thieno[2,3-d]pyrimidin-6-yl]-3-[1,3]dioxin-2-yl-propan-1-one in 1,4-dioxan, and the resulting solution was heated at ˜75° C. for ˜90 minutes. The resulting suspension was allowed cool and concentrated to a red solid. These solids were suspended in acetic acid and ammonium acetate (20 equivalents) added. The resulting suspension was heated at ˜125° C. for ˜24 hrs, allowed to cool to ambient temperature and poured into water. The mixture was extracted with ethyl acetate and the extracts washed with water then saturated aqueous sodium chloride solution. The organic phase was dried over anhydrous sodium sulphate and concentrated to a green-brown gum. The crude product was purified by column chromatography on silica gel, eluting with mixtures of ethyl acetate and hexane, to give the product as a yellow-green solid, dried in vacuo.

LC retention time 2.641 minutes [M+H]⁺ 360.9/362.9 (Run time 3.75 mins)

This compound had activity ‘A’ in the fluorescence polarization assay described below.

### EXAMPLE 22

### N-{5-[2-Amino-4-(2,4-dichloro-phenyl)-thieno[2,3-d]pyrimidin-6-yl]-4-methyl-1H-imidazol-2-yl}-acetamide

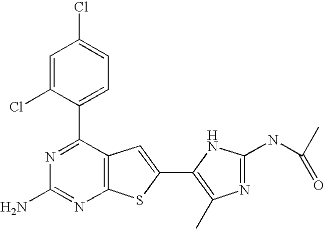

### Step 1

### 1-[2-amino-4-(2,4-dichloro-phenyl)-thieno[2,3-d]pyrimidin-6-yl]-2-bromo-propan-1-one

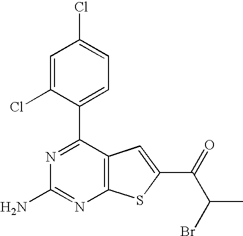

Copper (II) bromide was added to a suspension of 1-[2-Amino-4-(2,4-dichlorophenyl)thieno[2,3-d]pyrimidin-6-yl]-propan-1-one (example 8) in ethyl acetate under a nitrogen atmosphere. The resulting suspension was heated at ˜75° C., for 24 hrs. The resulting suspension was washed with water and then saturated aqueous sodium chloride solution. The solution was dried over anhydrous sodium sulphate and concentrated to an orange-brown solid. Solids were washed with hexane and dried in vacuo.

LC retention time 2.579 minutes [M+H]⁺ 429.75/431.8/433.7 (Run time 3.75 mins)

### Step 2

### N-{5-[2-Amino-4-(2,4-dichloro-phenyl)-thieno[2,3-d]pyrimidin-6-yl]-4-methyl-1H-imidazol-2-yl}-acetamide

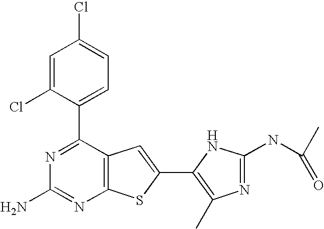

1-Acetylguanidine was added to a suspension of 1-[2-Amino-4-(2,4-dichlorophenyl)thieno[2,3-d]pyrimidin-6-yl]-2-bromo-propan-1-one in acetonitrile, under a nitrogen atmosphere. The resulting suspension was heated at ˜80° C., for ˜18 hrs. The resulting solution was allowed to cool and concentrated to an orange semi-solid. The crude product was purified by column chromatography, eluting with ethyl acetate, to give the product as an orange solid, solids were washed with diethyl ether and dried in vacuo.

LC retention time 2.225 minutes [M+H]⁺ 432.9/434.9 (Run time 3.75 mins)

This compound had activity ‘A’ in the fluorescence polarization assay described below.

### EXAMPLE 23

### 6-(2-amino-5-methyl-thiazol-4-yl)-4-(2,4-dichloro-phenyl)-thieno[2,3-d]pyrimidin-2-ylamine

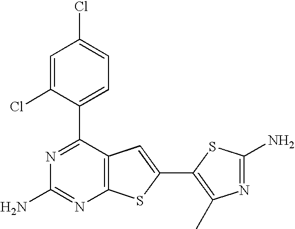

Thiourea was added to a suspension of 1-[2-Amino-4-(2,4-dichlorophenyl)thieno[2,3-d]pyrimidin-6-yl]-2-bromo-propan-1-one (example 22; step 1) in ethanol. The resulting suspension was heated at ˜95° C., for ˜90 minutes. The resulting solution was allowed to cool and concentrated to an orange gum. Water was added, and the resulting suspension made basic with saturated sodium hydrogen carbonate solution to give a pale orange solid suspension. Solids were removed by filtration washed with water and hexane, then dried in vacuo.

LC retention time 2.505 minutes [M+H]⁺ 407.9/409.8 (Run time 3.75 mins)

This compound had activity ‘A’ in the fluorescence polarization assay described below.

### EXAMPLE 24

### 4-(2,4-Dichloro-phenyl)-6-(3H-imidazol-4-yl)-thieno[2,3-d]pyrimidin-2-ylamine

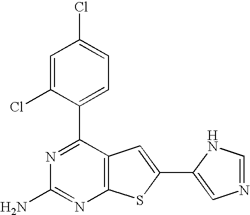

Toluene sulphonic acid was added to a suspension of 2-Amino-4-(2,4-dichloro-phenyl)-thieno[2,3-d]pyrimidine-6-carbaldehyde (example 8; step 1) and p-toluene sulphonamide in toluene and the mixture heated at ˜115° C., for ˜90 mins. The resulting solution was allowed to cool and concentrated to a brown solid. The solid was taken up in methanol/ethylene glycol dimethyl ether (2:1) and potassium carbonate was added, followed by p-tosyl methyl isocyanide and the mixture heated under reflux for ˜90 minutes. The suspension was allowed to cool and concentrated, dichloromethane was added to the residue and the mixture washed with water and saturated aqueous sodium chloride solution, Organic phase was dried over anhydrous sodium sulphate and concentrated to a brown gum. The crude product was purified by column chromatography on silica silica gel eluting with mixtures of dichloromethane and methanol. The crude product was re-purified by preparative HPLC, to give the product as a yellow solid.

LC retention time 1.996 minutes [M+H]⁺ 361.9/363.9 (Run time 3.75 mins)

This compound had activity ‘A’ in the fluorescence polarization assay described below.

### EXAMPLE 25

### 6-Aminomethyl-4-(2,4-dichloro-phenyl)-thieno[2,3-d]pyrimidin-2-ylamine

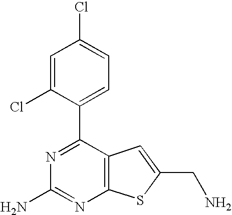

### Step 1

6-Chloromethyl-4-(2,4-dichloro-phenyl)-thieno[2,3-d]pyrimidin-2-ylamine hydrochloride

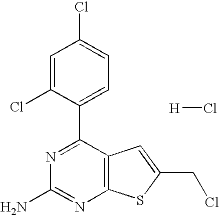

To a stirred suspension of [2-amino-4-(2,4-dichloro-phenyl)-thieno[2,3-d]pyrimidin-6-yl]methanol (example 6) (20 mg, 0.061 mmol) in toluene (1 mL) was added thionyl chloride (200 μL, 2.74 mmol). After 1.5 h, the solvent was removed in vacuo to afford a yellow powder which was used directly in the subsequent step.

¹H NMR (400 MHz; d⁶-DMSO) δ 5.01 (2H, s), 6.94 (1H, br s), 7.57 (1H, d, J=8 Hz), 7.60 (1H, dd, J=8 and 2 Hz), 7.84 (1H, d, J=2 Hz) and 6.6-7.1 broad shifted water peak.

### Step 2

### 6-Aminomethyl-4-(2,4-dichloro-phenyl)-thieno[2,3-d]pyrimidin-2-ylamine

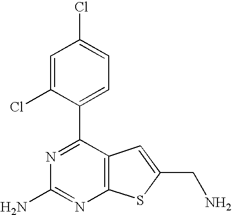

A mixture of 6-chloromethyl-4-(2,4-dichloro-phenyl)-thieno[2,3-d]pyrimidin-2-ylamine hydrochloride (25 mg, 0.066 mmol) and a solution of ammonia in methanol (7N; 3 mL) was stirred for one hour, then evaporated to afford a beige solid. Purification by reverse-phase preparative HPLC (standard method) afforded the desired product (5.5 mg) as a white solid.

LC-MS retention time: 1.70 min, [M+H]⁺ 325/327 (run time 3.75 mins)

¹H NMR (400 MHz; d⁶-DMSO) δ 3.84 (2H, br d, J=1 Hz), 6.62 (1H, br t, J=1 Hz), 6.85 (2H, br s), 7.52 (1H, d, J=8.3 Hz), 7.58 (1H, dd, J=8.3, 2.0 Hz) and 7.81 (1H, d, J=2.0 Hz).

This compound had activity ‘A’ in the fluorescence polarization assay described below.

### EXAMPLE 26

### N-[2-Amino-4-(2,4-dichloro-phenyl)-thieno[2,3-d]pyrimidin-6-ylmethyl]-acetamide

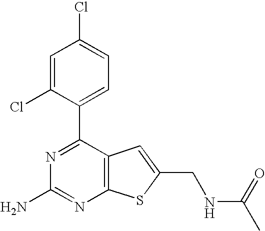

To a cloudy, stirred solution of 6-aminomethyl-4-(2,4-dichloro-phenyl)-thieno[2,3-d]pyrimidin-2-ylamine (example 25) (102 mg, 0.31 mmol) and triethylamine (87 μL, 0.63 mmol) in anhydrous DMF (4 mL) was added acetyl chloride (28 μL, 0.38 mmol) leading to a mild exotherm. After 2.25 h, the reaction mixture was filtered and solvent was removed in vacuo to afford the crude product. Purification by flash column chromatography [10 g SiO₂; 90:10 (ethyl acetate-hexane) ethyl acetate] afforded a yellow solid (35 mg) which was further purified by reverse-phase preparative HPLC (standard method) to afford the desired product (12 mg) as a white solid.

LC-MS retention time: 2.12 min, [M+H]⁺ 367/369 (run time 3.75 mins)

¹H NMR (400 MHz; d⁶-DMSO) δ 1.83 (3H, s), 4.35 (2H, d, J=6 Hz), 6.66 (1H, s), 6.96 (2H, br s), 7.53 (1H, d, J=8.3 Hz), 7.59 (1H, dd, J=8.3, 2.0 Hz), 7.83 (1H, d, J=2.0 Hz) and 8.47 (1H, t, J=6 Hz).

This compound had activity ‘A’ in the fluorescence polarization assay described below.

### EXAMPLE 27

### 4-(2,4-Dimethyl-phenyl)-thieno[2,3-d]pyrimidine-2-ylamine

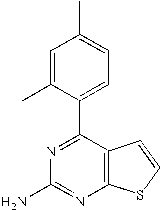

Quinoline (3 ml) was added to 2-Amino-4-(2,4-dimethyl-phenyl)-thieno[2,3-d]pyrimidine-6-carboxylic acid (example 2) (282 mg, 0.68 mmol) followed by copper powder (65 mg; 1.5 equiv). The reaction mixture was heated to 165-170° C. for 45 minutes (reaction mixture becomes very dark). The reaction mixture was allowed to cool to ambient temperature and was then added drop-wise to an ice bath cooled solution of aqueous Hydrochloric acid (1.2M; 50 mL). Ethyl acetate was added (50 mL) and reaction mixture was stirred vigorously for 5 mins. Mixture was then filtered through a pad of celite and filtrate phases were separated. The organic phase was washed with sat. aqueous sodium chloride solution, dried over Na₂SO₄ and evaporated to a brown oil which was purified by flash chromatography on silica gel (eluting with 0-50% ethyl acetate in hexane), then further purified by preparative HPLC to afford product as off white solid (30 mg).

LC-MS retention time: 2.522 min, [M+H]⁺ 256 (run time 3.75 mins)

This compound had activity ‘A’ in the fluorescence polarization assay described below.

### EXAMPLE 28

### 2-amino-4-(5-benzyloxy-2,4-dichloro-phenyl)-thieno[2,3-d]pyrimidine-6-carbonitrile

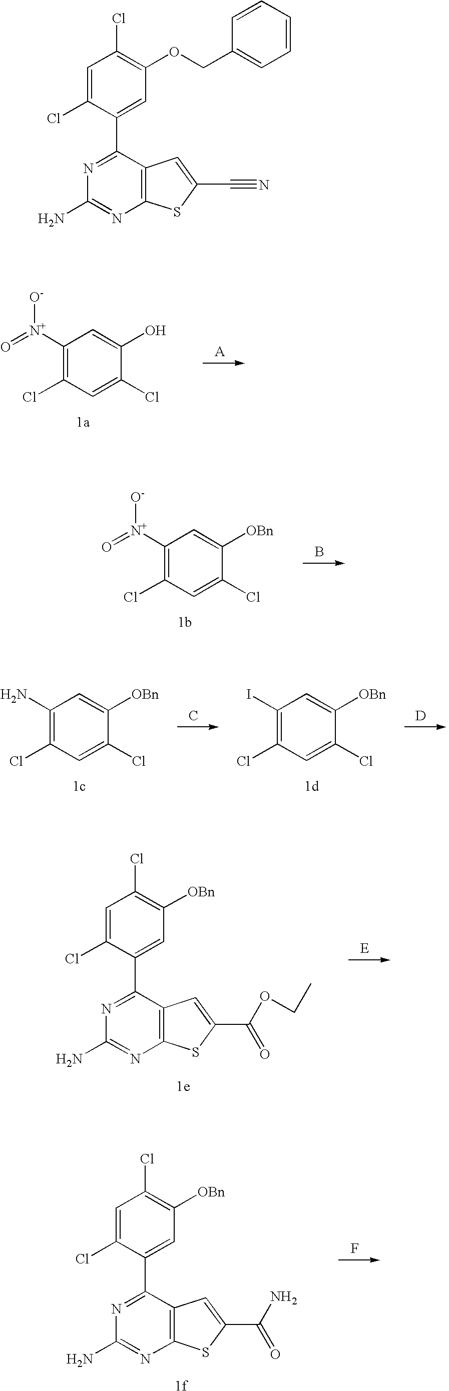

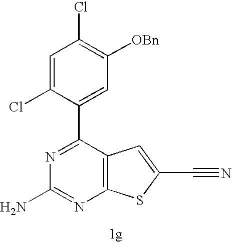

### Step 1

### 1-Benzyloxy-2,4-dichloro-5-nitro-benzene

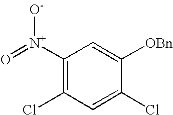

Potassium carbonate (12 g, 87 mmol) was added to a solution of 2,4-dichloro-5-nitrophenol (Lancaster Synthesis, Morecambe, Lancashire, UK) (15.6 g, 75 mmol) in acetone. Benzyl bromide (9 ml, 76 mmol) was added and the suspension heated at 75° C. (oil bath temperature) for ˜3 hrs. The resulting suspension was allowed to cool and water (500 ml) was added, the mixture was extracted with dichloromethane (2×200 ml). The combined extracts were washed with aqueous sodium hydroxide (150 ml, 2M), water (2×200 ml) and saturated aqueous sodium chloride solution (150 ml). The solution was dried over anhydrous sodium sulphate and concentrated to a pale yellow solid (21.5 g, 96%)

### R_{f} 0.73 CH₂Cl₂ (SiO₂)

LC retention time 2.915 min [M+H]+no ionisation (run time 3.75 min)

### Step 2

### 5-Benzyloxy-2,4-dichloro-phenylamine

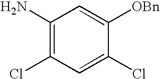

Iron powder (21 g, 376 mmol) was added to a suspension 1-Benzyloxy-2,4-dichloro-5-nitro-benzene (21.5 g, 72 mmol) in acetic acid (300 ml)/water (150 ml) and the mixture was heated at 85° C. (oil bath temperature) for ˜90 mins. The resulting suspension was filtered. The filtrate was allowed to cool, water (750 ml) was added and the mixture extracted with dichloromethane (3×150 ml). The combined extracts were washed with aqueous sodium hydroxide (300 ml, 2M), water (2×500 ml) and saturated aqueous sodium chloride solution (200 ml). The solution was dried over anhydrous sodium sulphate filtered and the filtrate solvents removed in vacuo to afford product as a pale brown solid (18.6 g, 96%)

### R_{f} 0.57 CH₂Cl₂ (SiO₂)

LC retention time 2.792 min [M+H]⁺ 270/268 (run time 3.75 min)

### Step 3

### 1-Benzyloxy-2,4-dichloro-5-iodo-benzene

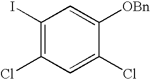

Hydrochloric acid (60 ml, 6M) was added to a solution of the 5-Benzyloxy-2,4-dichloro-phenylamine (16.2 g, 60 mmol) in acetic acid (240 ml) and the resulting suspension cooled (ice/water/salt). Aqueous sodium nitrite (4.8 g, 69.5 mmol in 40 ml) was added slowly (keeping the temperature <5° C.). On complete addition the resulting solution was stirred for ˜30 mins.

The resulting solution was poured into a solution of potassium iodide (20 g, 120 mmol) and iodine (4 g, 16 mmol) in water (200 ml), and the mixture stirred for ˜90 mins. Water (800 ml) was added and the mixture extracted with dichloromethane (3×250 ml). The combined extracts were washed with aqueous sodium thiosulphate solution (2×150 ml, 10%), aqueous sodium hydroxide (250 ml, 2M), water (2×250 ml) and saturated aqueous sodium chloride solution (200 ml). The solution was dried over anhydrous sodium sulphate and concentrated to a pale brown oil, solidified on standing. (20.6 g, 90%)

### R_{f} 0.82 CH₂Cl₂ (SiO₂)

LC retention time 3.084 min [M+H]⁺ no ionisation (run time 3.75 min)

### Step 4

### 2-Amino-4-(5-benzyloxy-2,4-dichloro-phenyl)-thieno[2,3-d]pyrimidine-6-carboxylic acid ethyl ester

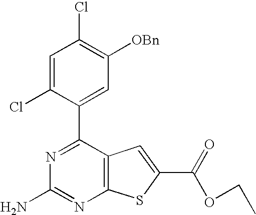

Potassium acetate (16 g, 163 mmol) was added to a solution of 1-Benzyloxy-2,4-dichloro-5-iodo-benzene (20.6 g, 54 mmol) and bis(pinacolato)diboron (14.5 g, 57 mmol) in DMF (50 ml), under a nitrogen atmosphere. Palladium acetate (450 mg, cat.) was added and the mixture heated, oil bath temperature 90° C., for ˜18 hrs. The resulting solution was concentrated, and the residue taken up in ethyl acetate (200 ml), the solution was washed with water (3×200 ml) and saturated aqueous sodium chloride solution (150 ml). The solution was dried over anhydrous sodium sulphate and concentrated to a pale brown gum.

The residue was taken up in 1,4-dioxan (160 ml) and 2-Amino-4-chloro-thieno[2,3-d]pyrimidine-6-carboxylic acid ethyl ester (example 1; step 1) (12.85 g, 50 mmol) and aqueous potassium phosphate (40 ml, 2M) added, under a nitrogen atmosphere. Dichloro bis(triphenylphosphine)palladium(II) (cat.) was added and the mixture heated, oil bath temperature 100° C., for ˜3 hrs. The mixture was allowed to cool and ethyl acetate (400 ml) added. The mixture was washed with saturated aqueous sodium chloride solution (100 ml). The solution was dried over anhydrous sodium sulphate and concentrated to a pale yellow solid. Solids were washed with diethyl ether/hexane (1:1), to give an off-white solid. Dried in vacuo (40° C.). 10.7 g (45%) R_{f} 0.13 EtOAc/Hex (1:3) (SiO₂)

LC retention time 2.972 min [M+H]⁺ 476/474 (run time 3.75 min)

### Step 5

### 2-amino-4-(5-benzyloxy-2,4-dichloro-phenyl)-thieno[2,3-d]pyrimidine-6-carbonitrile

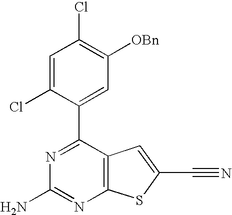

A suspension of 2-Amino-4-(5-benzyloxy-2,4-dichlorophenyl)-thieno[2,3-d]pyrimidine-6-carboxylic acid ethyl ester in methanolic ammonia (˜7N) was heated, sealed tube at ˜85° C., for ˜72 hrs. The resulting solution was concentrated and the residue triturated with diethyl ether to give a pale yellow powder.

Trifluoroacetic anhydride was added to a solution of 2-Amino-4-(5-benzyloxy-2,4-dichlorophenyl)-thieno[2,3-d]pyrimidine-6-carboxylic acid amide in pyridine/dichloromethane, at ˜0° C. (ice/water), and the solution stirred for ˜2 hrs. Water was added and the mixture washed with aqueous ammonia (0.880), water and saturated aqueous sodium chloride solution. Solution was dried over anhydrous sodium sulphate and concentrated. The crude product was purified by column chromatography on silica gel, eluting with mixtures of ethyl acetate and hexane, to give the product as a yellow solid.

LC retention time 2.836 minutes [M+H]⁺ 426.9/428.9 (Run time 3.75 mins)

This compound had activity ‘A’ in the fluorescence polarization assay described below.

### EXAMPLE 29

### 2-Amino-4-[2,4-dichloro-5-(diethylamino-ethoxy)-phenyl]-thieno[2,3-d]pyrimidine-6-carbonitrile

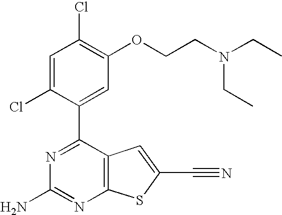

### Step 1

### 2-Amino-4-(2,4-dichloro-5-hydroxy-phenyl)-thieno[2,3-d]pyrimidine-6-carbonitrile

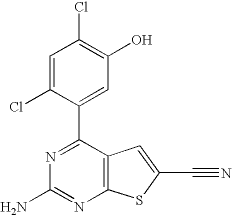

Boron trichloride (1M in dichloromethane) was added slowly to a suspension of 2-Amino-4-(5-benzyloxy-2,4-dichlorophenyl)-thieno[2,3-d]pyrimidine-6-carbonitrile in dichloromethane at −78° C. (dry ice/acetone) under a nitrogen atmosphere. The mixture was stirred for ˜1 hr. at −78° C., and for ˜3 hrs. at room temperature. The mixture was cooled (ice/water) and methanol was added slowly, the resulting solution was stirred for ˜1 hr. at room temperature. The solution was concentrated to a brown gum; and the residue was taken up in methanol and concentrated to give a brown gum. The crude product was purified by column chromatography on silica gel eluting with mixtures of dichloromethane and methanol to give a pale yellow solid.

LC retention time 2.411 minutes [M+H]⁺ 336.9/338.9 (Run time 3.75 mins)

### Step 2

### 2-Amino-4-[2,4-dichloro-5-(diethylamino-ethoxy)-phenyl]-thieno[2,3-d]pyrimidine-6-carbonitrile

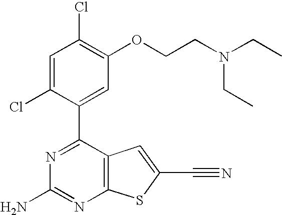

Cesium carbonate was added to a solution of 2-Amino-4-(2,4-dichloro-5-hydroxyphenyl)-thieno[2,3-d]pyrimidine-6-carbonitrile in DMF, 2-Bromo-N,N-diethylethylamine hydrobromide was added and the suspension heated, at ˜140° C., for ˜60 mins. The resulting suspension was allowed to cool and dichloromethane added. The mixture was washed with water and saturated aqueous sodium chloride solution. The solution was dried over anhydrous sodium sulphate and concentrated to a dark brown gum. The crude product was purified by chromatography eluting with ethyl acetate to give a pale brown gum, trituration with hexane gave a pale yellow solid.

LC retention time 1.994 minutes [M+H]⁺ 436/438 (Run time 3.75 mins)

This compound had activity ‘A’ in the fluorescence polarization assay described below.

### EXAMPLE 30

### 2-Amino-4-[2,4-dichloro-5-(2-diethylamino-ethoxy)-phenyl]-N-hydroxy-thieno[2,3-d]pyrimidine-6-carboxamidine

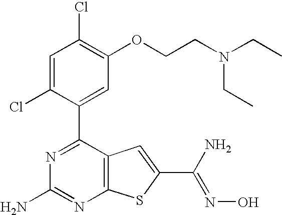

Hydroxylamine hydrochloride and sodium acetate were added to a suspension of 2-Amino-4-[2,4-dichloro-5-(2-diethylamino-ethoxy)-phenyl]-thieno[2,3-d]pyrimidine-6-carbonitrile (example 29) in ethanol and the suspension heated at 80° C. for ˜90 minutes. The resulting suspension was allowed to cool and water added to give a pale yellow precipitate. Solids were removed by filtration and washed with water and hexane, to give a pale yellow powder.

LC retention time 1.730 minutes [M+H]⁺ 469/471 (Run time 3.75 mins)

This compound had activity ‘A’ in the fluorescence polarization assay described below.

### EXAMPLE 31

### 4-[2,4-dichloro-5-(2-diethylamino-ethoxy)-phenyl]-6-(4,5-dihydro-1H-imidazol-2-yl)-thieno[2,3-d]pyrimidin-2-ylamine

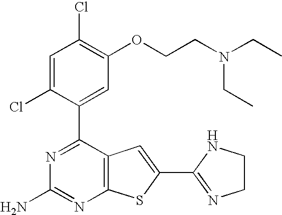

Ethylene diamine (10 eq) and acetic acid (20 eq) were added to a solution of 2-Amino-4-[2,4-dichloro-5-(2-diethylamino-ethoxy)-phenyl]-N-hydroxy-thieno[2,3-d]pyrimidine-6-carboxamidine (example 30) in ethanol and the solution heated, ˜125° C. sealed tube, for ˜18 hrs. The resulting solution was allowed to cool and concentrated to a pale brown semi-solid. The residue purified by preparative HPLC.

LC retention time 2.283 minutes [M+H]⁺ 479/481 (Run time 7.00 mins)

This compound had activity ‘A’ in the fluorescence polarization assay described below.

### EXAMPLE 32

### {2-amino-4-[2,4-dichloro-5-(2-diethylamino-ethoxy)-phenyl]-thieno[2,3-d]pyrimidin-6-yl}-methanol

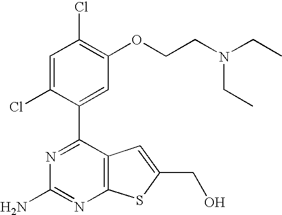

### Step 1

### 2-amino-4-(2,4-dichloro-5-hydroxy-phenyl)-thieno[2,3-d]pyrimidin-6-carboxylic acid ethyl ester

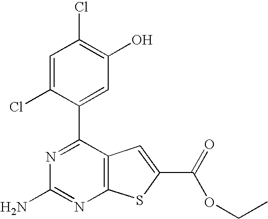

Boron trichloride (1M solution in dichloromethane) was added slowly to a suspension of 2-Amino-4-(5-benzyloxy-2,4-dichlorophenyl)-thieno[2,3-d]pyrimidine-6-carboxylic acid ethyl ester in dichloromethane at −78° C. (dry ice/acetone) under a nitrogen atmosphere. The mixture was stirred for ˜1 hr. at −78° C., and for ˜3 hrs. at room temperature. The mixture was cooled (ice/water) and methanol was added slowly, the resulting solution was then stirred for ˜1 hr. at room temperature. The solution was concentrated to a brown gum, the residue was taken up in methanol and concentrated to give a brown gum. The crude product was purified by column chromatography eluting with mixtures of dichloromethane and methanol to give a pale yellow solid.

LC retention time 2.559 minutes [M+H]⁺ 383.9/385.9 (Run time 3.75 mins)

Step 2

### 2-Amino-4-(2,4-dichloro-5-(2-diethylamino-ethoxy)-phenyl)-thieno[2,3-d]pyrimidine-6-carboxylic acid ethyl ester

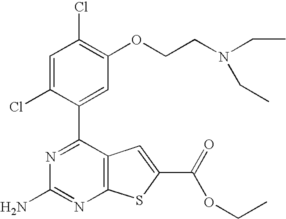

Cesium carbonate was added to a solution of 2-Amino-4-(2,4-dichloro-5-hydroxyphenyl)-thieno[2,3-d]pyrimidine-6-carboxylic acid ethyl ester in DMF, 2-bromo-N,N-diethylethylamine hydrobromide was added and the suspension heated, at ˜140° C., for ˜90 minutes. The resulting suspension was allowed to cool to ambient temperature and dichloromethane added. The mixture was washed with water and saturated aqueous sodium chloride solution. The solution was dried over anhydrous sodium sulphate and concentrated to a dark brown gum. The crude product was purified by chromatography on silica gel eluting with mixtures of dichloromethane and methanol to give a pale brown solid.

LC retention time 2.026 minutes [M+H]⁺ 483/485 (Run time 3.75 mins)

### Step 3

### {2-amino-4-[2,4-dichloro-5-(2-diethylamino-ethoxy)-phenyl]-thieno[2,3-d]pyrimidin-6-yl}-methanol

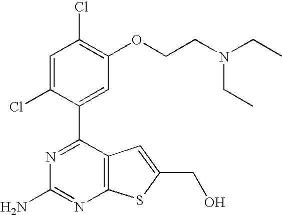

Diisobutylaluminium hydride (1M in THF) was added slowly to a solution of 2-Amino-4-[2,4-dichloro-5-(2-diethylamino-ethoxy)-phenyl]-thieno[2,3-d]pyrimidine-6-carboxylic acid ethyl ester in THF at −78° C. (dry ice/acetone) under a nitrogen atmosphere. The mixture was stirred for ˜1 hr. at −78° C., and for ˜90 mins. at room temperature. The mixture was cooled (ice/water) and methanol was added slowly, the resulting solution was stirred for ˜1 hr. at room temperature. The solution was concentrated to a brown gum. The residue was suspended in saturated aqueous sodium chloride solution and extracted with ethyl acetate, the combined extracts were dried over anhydrous sodium sulphate and concentrated to a brown gum. The crude product was purified by column chromatography on silica gel eluting with mixtures of dichloromethane and methanol to give a pale brown foam.

LC retention time 1.728 minutes [M+H]⁺ 440.95/442.9 (Run time 3.75 mins)

This compound had activity ‘A’ in the fluorescence polarization assay described below.

### EXAMPLE 33

### 2-amino-4-[2,4-dicholoro-5-(2-diethylamino-ethoxy)-phenyl]thieno[2,3-d]pyrimidine-6-carbaldehyde

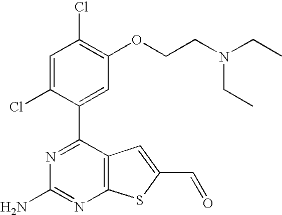

Manganese (IV) oxide (15 equivalents) was added to a solution of {2-Amino-4-[2,4-dichloro-5-(2-diethylamino-ethoxy)-phenyl]-thieno[2,3-d]pyrimidin-6-yl}-methanol (example 32) in ethylene glycol dimethyl ether and the mixture stirred for ˜60 hrs. The resulting suspension was filtered and the solids washed with ethylene glycol dimethyl ether, the combined filtrates were concentrated to a yellow/brown solid. Solids were washed with hexane and dried in vacuo.

LC retention time 1.871 minutes [M+H]⁺ 438.9/440.9 (Run time 3.75 mins)

This compound had activity ‘A’ in the fluorescence polarization assay described below.

### EXAMPLE 34

### 4-{2-Amino-4-[2,4-dicholoro-5-(2-diethylamino-ethoxy)-phenyl]thieno[2,3-d]pyrimidine-6-yl}-but-3-en-2-one

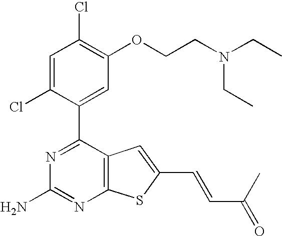

1-(Triphenylphosphoranylidene)-2-propanone was added to a suspension of 2-Amino-4-[2,4-dichloro-5-(2-diethylamino-ethoxy)-phenyl]-thieno[2,3-d]pyrimidine-6-carbaldehyde (example 33) in toluene and the mixture heated at ˜85° C., for ˜90 minutes. The resulting suspension was allowed to cool and concentrated to a yellow gum. The crude product was purified by column chromatography eluting with mixtures of dichloromethane and methanol to give the product as a yellow gum, trituration with hexane and dried in vacuo to give the product as a yellow solid.

LC retention time 1.965 minutes [M+H]⁺ 479/481 (Run time 3.75 mins)

This compound had activity ‘A’ in the fluorescence polarization assay described below.

### EXAMPLE 35

### 4-[2,4-dicholoro-5-(2-diethylamino-ethoxy)-phenyl]-6-(5-methyl-2H-pyrazol-3-yl)-thieno[2,3-d]pyrimidine-2-ylamine

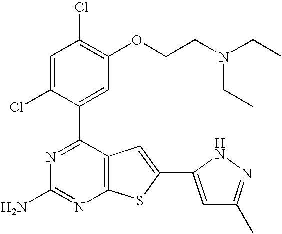

Para-toluene sulphonylhydrazide and sodium ethoxide were added to a suspension of 4-{2-Amino-4-[2,4-dichloro-5-(2-diethylamino-ethoxy)-phenyl]-thieno[2,3-d]pyramid-6-yl}-but-3-en-2-one (example 34) in ethanol, under a nitrogen atmosphere, and the mixture heated at ˜80° C., for ˜18 hrs. The resulting solution was allowed to cool and concentrated to an orange solid. The crude product was purified by preparative HPLC, to give the product as a yellow solid.

LC retention time 1.917 minutes [M+H]⁺ 491/493 (Run time 3.75 mins)

This compound had activity ‘A’ in the fluorescence polarization assay described below.

### EXAMPLE 36

### 2-Amino-4-[2,4-dichloro-5-(2-diethylamino-ethoxy)-phenyl]-thieno[2,3-d]pyrimidine-6-carboxylic acid

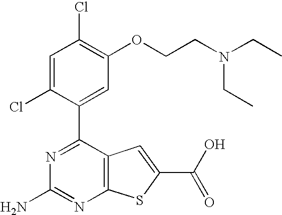

This compound was made from 2-Amino-4-(2,4-dichloro-5-(2-diethylamino-ethoxy)-phenyl)-thieno[2,3-d]pyrimidine-6-carboxylic acid ethyl ester (step 2; example 32) by way of the method of example 1 step 3.

LC retention time 1.74 minutes [M+H]⁺ 455/457 (Run time 3.75 mins)

This compound had activity ‘A’ in the fluorescence polarization assay described below.

### EXAMPLE 37

### 2-Amino-4-[5-benzyloxy-2,4-dichloro-phenyl]-thieno[2,3-d]pyrimidine-6-carboxylic acid

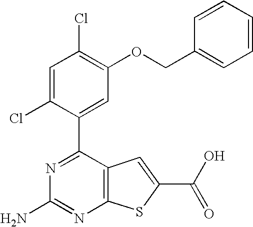

This compound was made from 2-Amino-4-(5-benzyloxy-2,4-dichloro-phenyl)-thieno[2,3-d]pyrimidine-6-carboxylic acid ethyl ester (example 28; step 4) by way of the method of example 1 step 3).

This compound had activity ‘A’ in the fluorescence polarization assay described below.

### EXAMPLE 38

### 6-(5-Amino-1H-[1,2,4]triazol-3-yl)-4-(2,4-dichloro-phenyl)-thieno[2,3-d]pyrimidin-2-ylamine

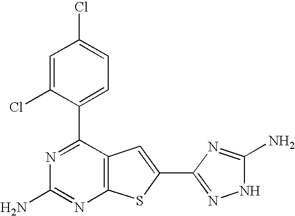

### Step 1

### 2-Amino-4-(2,4-dichloro-phenyl)-thieno[2,3-d]pyrimidine-6-carboxylic acid hydrazide

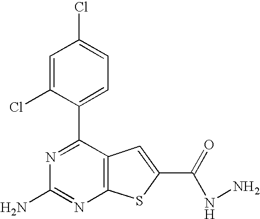

Hydrazine hydrate was added to a suspension of 2-Amino-4-(2,4-dichloro-phenyl)-thieno[2,3-d]pyrimidine-6-carboxylic acid ethyl ester (example 1; step 2) in ethanol and the mixture heated under reflux for ˜3 hrs. The resulting suspension was allowed to cool and concentrated to a brown solid. The solids were washed with water and hexane, to give a pale brown powder.

LC retention time 1.954 minutes [M+H]⁺ 353.9/355.9 (Run time 3.75 mins)

### Step 2

### 6-(5-Amino-1H-[1,2,4]triazol-3-yl)-4-(2,4-dichloro-phenyl)-thieno[2,3-d]pyrimidin-2-ylamine

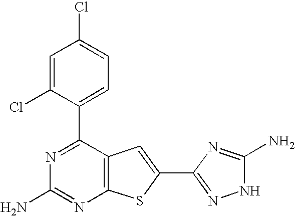

S-Methylisothiourea sulphate was added to a suspension of 2-Amino-4-(2,4-dichloro-phenyl)-thieno[2,3-d]pyrimidine-6-carboxylic acid hydrazide and sodium acetate in pyridine and the mixture heated at ˜110° C., for ˜18 hrs. The resulting suspension was allowed to cool and concentrated to a yellow-orange solid. The solids were taken up in ethyl acetate washed with water and saturated aqueous sodium chloride solution. The solution was dried over anhydrous sodium sulphate and concentrated to give a pale brown powder. The crude product was purified by column chromatography, silica, eluting with ethyl acetate to give the product as an orange-brown foam. Trituration with diethyl ether gave an orange-brown powder. Dried in vacuo.

LC retention time 1.978 minutes [M+H]⁺ 377.9/379.9 (Run time 3.75 mins)

This compound had activity ‘A’ in the fluorescence polarization assay described below.

### EXAMPLE 39

### 6-(5-Amino-1H-[1,2,4]triazol-3-yl)-4-[2,4-dichloro-5-(2-diethylamino-ethoxy)-phenyl]-thieno[2,3-d]pyrimidin-2-ylamine

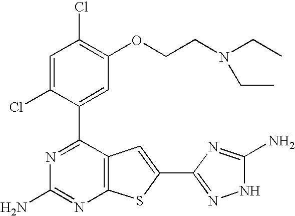

This compound was made from 2-Amino-4-(2,4-dichloro-5-(2-diethylamino-ethoxy)-phenyl)-thieno[2,3-d]pyrimidine-6-carboxylic acid ethyl ester, by the method of example 38.

LC retention time 2.314 minutes [M+H]⁺ 493/495 (Run time 7.00 mins)

This compound had activity ‘A’ in the fluorescence polarization assay described below.

### EXAMPLE 40

### 4-[2,4-Dichloro-5-(2-diethylamino-ethoxy)-phenyl]-6-(2H-tetrazol-5-yl)-thieno[2,3-d]pyrimidin-2-ylamine

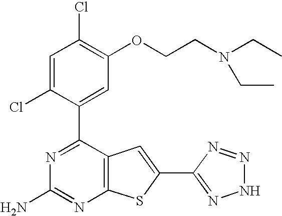

Sodium azide and ammonium chloride were added to a solution of the 2-Amino-4-[2,4-dichloro-5-(2-diethylamino-ethoxy)-phenyl]-thieno[2,3-d]pyrimidine-6-carbonitrile (example 29) in DMF. The mixture heated at ˜125° C., for ˜90 mins, the resulting suspension was allowed to cool and concentrated to a brown gum. Trituration with water gave a brown precipitate. Solids were removed by filtration and purified by preparative HPLC.

LC retention time 2.432 minutes [M+H]⁺ 479/481 (Run time 7.0 mins)

This compound had activity ‘A’ in the fluorescence polarization assay described below.

### EXAMPLE 41

### 3-{2-Amino-4-[2,4-Dichloro-5-(2-diethylamino-ethoxy)-phenyl]-thieno[2,3-d]pyrimidin-6-yl}-acrylic acid ethyl ester

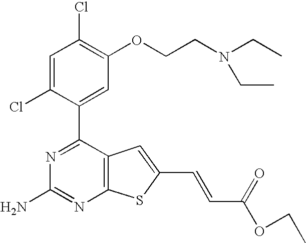

Potassium carbonate was added to a solution of 2-Amino-4-(2,4-dichloro-5-(2-diethylamino-ethoxy)-phenyl)-thieno[2,3-d]pyrimidine-6-carbaldehyde (example 33) and triethyl phosphonoacetate in acetonitrile the mixture heated at ˜85° C., for ˜18 hrs. The resulting suspension was allowed to cool and concentrated to a yellow solid. Dichloromethane was added and the mixture washed with water and saturated aqueous sodium chloride solution. The solution was dried over anhydrous sodium sulphate and concentrated to a pale yellow solid. The crude product was purified by column chromatography, silica, eluting with mixtures of dichloromethane and methanol to give the product as yellow/brown foam. Trituration with diethyl ether gave a pale yellow powder, which was dried in vacuo.

LC retention time 2.047 minutes [M+H]⁺ 509/511 (Run time 3.75 mins)

This compound had activity ‘A’ in the fluorescence polarization assay described below.

### EXAMPLE 42

### 4-(2,4-dichloro-phenyl)-6-nitro-thieno[2,3-d]pyrimidin-2-ylamine

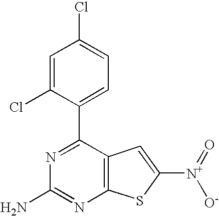

### Step 1

### 2-Amino-4-chloro-6-(2,4-dicholoro)-pyrimidine-5-carbaldehyde

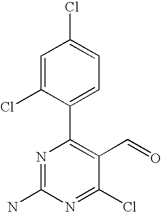

2-Amino-4,6-dichloro-pyridinecarbaldehyde 1 g (5.21 mmols, 1 eq) and 2,4-dichlorophenylboronic acid 1.04 g (5.47 mmols, 1.05 eqs) were dissolved in 15 mL of 1,4-dioxane. Nitrogen was bubbled through the solution whilst aqueous potassium phosphate (0.85 g in 4 mL H₂O) was added. The solution had nitrogen bubbled through it for a further 4 minutes. Bis(triphenylphosphine)palladium(II) chloride (20 mg) was added and nitrogen passed through the solution for a further minute. This was then heated to reflux under nitrogen for 12 hours. The solution was cooled to ambient temperature and then diluted with brine and basified by adding 10 mL 2M NaOH. Extracted 3 times with ethyl acetate and dried over MgSO₄. The solvents were removed under vacuum leaving a yellow oil. This contained a significant amount of disubstituted material. Dichloromethane was added and product was partially soluble, methanol was added slowly to this and a pale yellow precipitate formed which was filtered off and dried under vacuum. Giving a 38% yield with a trace of disubstituted product.

LC-MS retention time minutes 2.385 [M+H]⁺=301.9+303.9 (run time 3.75 minutes)

### Step 2

### 4-(2,4-dichloro-phenyl)-6-nitro-thieno[2,3-d]pyrimidin-2-ylamine

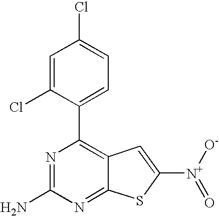

Sodium sulphide 1.55 g (19.8 mmols, 1.2 eq) and sulphur (19.8 mmols, 1.2 eq) were added to dimethylformamide (50 mL) under nitrogen. The solution immediately turned dark blue. After 20 minutes 2-Amino-4-chloro-6-(2,4-dichlorophenyl)-pyrimidine-5-carbaldehyde 5 g (16.5 mmols, 1 eq) was added portion wise at 70° C. with heating, this was stirred under nitrogen for 3.5 hours, this now yellow solution was cooled to ambient temperature and bromonitromethane was added 1.15 mL (16.5 mmols, 1 eq) generating some heat. This was then heated to 65° C. for 1.5 hours, followed by the addition of sodium methoxide 891 mg (16.5 mmol, 1 eq) at the same temperature. This was stirred for a further 30 minutes, cooled to ambient temperature and water was then added forming yellow precipitate which was filtered off and purified by flash column chromatography eluting dichloromethane to 5% methanol/dichloromethane giving the product as a yellow powder in 21% yield. This was further purified using preparative HPLC at pH 4.

LC-MS retention time minutes 2.665 [M+H]⁺=340.9+342.9 (run time 3.75 minutes)

### Fluorescence Polarization Assay

Fluorescence polarization {also known as fluorescence anisotropy} measures the rotation of a fluorescing species in solution, where the larger molecule the more polarized the fluorescence emission. When the fluorophore is excited with polarized light, the emitted light is also polarized. The molecular size is proportional to the polarization of the fluorescence emission.

The fluoroscein-labelled probe —RBT0051001-FAM—

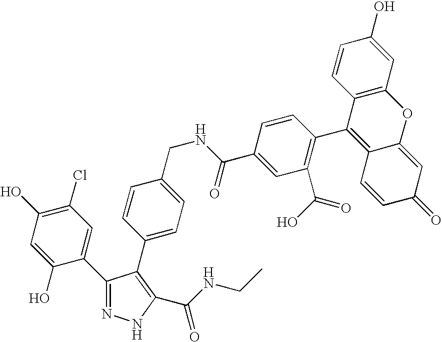

binds to HSP90 {full-length human, full-length yeast or N-terminal domain HSP90} and the anisotropy {rotation of the probe:protein complex} is measured.

Test compound is added to the assay plate, left to equilibrate and the anisotropy measured again. Any change in anisotropy is due to competitive binding of compound to HSP90, thereby releasing probe.

### Materials

Chemicals are of the highest purity commercially available and all aqueous solutions are made up in AR water.
1) Costar 96-well black assay plate #3915
2) Assay buffer of (a) 100 mM Tris pH7.4; (b) 20 mM KCl; (c) 6 mM MgCl₂. Stored at room temperature.
3) BSA (bovine serum albumen) 10 mg/ml (New England Biolabs # B9001S)
4) 20 mM probe in 100% DMSO stock concentration. Stored in the dark at RT. Working concentration is 200 nM diluted in AR water and stored at 4° C. Final concentration in assay 80 nM.
5) E. coli expressed human full-length HSP90 protein, purified >95% (see, e.g., Panaretou et al., 1998) and stored in 50 μL aliquots at −80° C.

### Protocol

1) Add 100 μl 1× buffer to wells 11A and 12A (=FP BLNK)
2) Prepare assay mix—all reagents are kept on ice with a lid on the bucket as the probe is light-sensitive.

i. Final Concⁿ

| 1× Hsp90 FP Buffer | 10 | ml | 1× | |
|---|---|---|---|---|
| BSA 10 mg/ml (NEB) | 5.0 | μl | 5 | μg/ml |
| Probe 200 μM | 4.0 | μl | 80 | nM |
| Human full-length Hsp90 | 6.25 | μl | 200 | nM |

3) Aliquot 100 μl assay mix to all other wells
4) Seal plate and leave in dark at room temp for 20 minutes to equilibrate
   Compound Dilution Plate—1×3 dilution series
1) In a clear 96-well v-bottom plate-{# VWR 007/008/257} add 10 μl 100% DMSO to wells B1 to H11
2) To wells A1 to A11 add 17.5 μl 100% DMSO
3) Add 2.5 μl cpd to A1. This gives 2.5 mM {50×} stock cpd-assuming cpds 20 mM.
4) Repeat for wells A2 to A10. Control in columns 11 and 12.
5) Transfer 5 μl from row A to row B—not column 12. Mix well.
6) Transfer 5 μl from row B to row C. Mix well.
7) Repeat to row G.
8) Do not add any compound to row H—this is the 0 row.
9) This produces a 1×3 dilution series from 50 μM to 0.07 μM.
10) In well B12 prepare 20 μl of 100 μM standard compound.
11) After first incubation the assay plate is read on a Fusion™ α-FP plate reader (Packard BioScience, Pangbourne, Berkshire, UK).
12) After the first read, 2 μl of diluted compound is added to each well for columns 1 to 10. In column 11 {provides standard curve} only add compound B11-H11. Add 2 μl of 100 mM standard cpd to wells B12-H12 {is positive control}
13) The Z′ factor is calculated from zero controls and positive wells. It typically gives a value of 0.7-0.9.

The compounds tested in the above assay were assigned to one of two activity ranges, namely A=<10 μM; B=>10 μM, and those assignments are reported above.

A growth inhibition assay was also employed for the evaluation of candidate HSP90 inhibitors:

Assessment of cytotoxicity by Sulforhodamine B (SRB) assay: calculation of 50% inhibitory concentration (IC₅₀).

### Day 1

1) Determine cell number by haemocytometer.
2) Using an 8 channel multipipettor, add 160 μl of the cell suspension (3600 cells/well or 2×10⁴ cells/ml) to each well of a 96-well microtitre plate.
3) Incubate overnight at 37° C. in a CO₂ incubator.

### Day 2

4) Stock solutions of drugs are prepared, and serial dilutions of each drug are performed in medium to give final concentrations in wells.
5) Using a multipipettor, 40 μl of drug (at 5× final concentration) is added to quadruplicate wells.
6) Control wells are at either side of the 96 well plates, where 40 μl of medium is added.
7) Incubate plates in CO₂ incubator for 4 days (48 hours).

### Day 6

8) Tip off medium into sink and immerse plate slowly into 10% ice cold trichloroacetic acid (TCA). Leave for about 30 mins on ice.
9) Wash plates three times in tap water by immersing the plates into baths of tap water and tipping it off.
10) Dry in incubator.
11) Add 100 μl of 0.4% SRB in 1% acetic acid to each well (except the last row (right hand) of the 96 well plate, this is the 0% control, ie no drug, no stain. The first row will be the 100% control with no drug, but with stain). Leave for 15 mins.
12) Wash off unbound SRB stain with four washes of 1% acetic acid.
13) Dry plates in incubator.
14) Solubilise SRB using 100 μl of 10 mM Tris base and put plates on plate shaker for 5 mins.
15) Determine absorbance at 540 nm using a plate reader. Calculate mean absorbance for quadruplicate wells and express as a percentage of value for control, untreated wells.
16) Plot % absorbance values versus log drug concentration and determine the IC₅₀.
   By way of illustration, the compound of Example 10 had an IC50<50 uM in the SRB growth arrest assay.

### REFERENCES

A number of publications are cited above in order to more fully describe and disclose the invention and the state of the art to which the invention pertains. Full citations for these references are provided below. Each of these references is incorporated herein by reference in its entirety into the present disclosure.
Argon Y and Simen B B. 1999 “Grp94, an ER chaperone with protein and peptide binding properties”, Semin. Cell Dev. Biol., Vol. 10, pp. 495-505.
Bijlmakers M-J J E, Marsh M. 2000 “Hsp90 is essential for the synthesis and subsequent membrane association, but not the maintenance, of the Src-kinase p56Ick”, Molecular Biology of the Cell, Vol. 11 (5), pp. 1585-1595.
Bucci M; Roviezzo F; Cicala C; Sessa W C, Cirino G. 2000 “Geldanamycin, an inhibitor of heat shock protein 90 (Hsp90) mediated signal transduction has anti-inflammatory effects and interacts with glucocorticoid receptor in vivo”, Brit. J. Pharmacol., Vol 131(1), pp. 13-16.
Chen C-F, Chen Y, Dai K D, Chen P-L, Riley D J and Lee W-H. 1996 “A new member of the hsp90 family of molecular chaperones interacts with the retinoblastoma protein during mitosis and after heat shock”, Mol. Cell. Biol., Vol. 16, pp. 4691-4699.
Chiosis G, Timaul M N, Lucas B, Munster P N, Zheng F F, Sepp-Lozenzino L and Rosen N. 2001 “A small molecule designed to bind to the adenine nucleotide pocket of HSP90 causes Her2 degradation and the growth arrest and differentiation of breast cancer cells”, Chem. Biol., Vol. 8, pp. 289-299.
Conroy S E and Latchman D S. 1996 “Do heat shock proteins have a role in breast cancer?”, Brit. J. Cancer, Vol. 74, pp. 717-721.
Felts S J, Owen B A L, Nguyen P, Trepel J, Donner D B and Toft D O. 2000 “The HSP90-related protein TRAP1 is a mitochondrial protein with distinct functional properties”, J. Biol. Chem., Vol. 5, pp. 3305-3312.
Fuller W, Cuthbert A W. 2000 “Post-translational disruption of the delta F508 cystic fibrosis transmembrane conductance regulator (CFTR)-molecular Chaperone complex with geldanamycin stabilizes delta F508 CFTR in the rabbit reticulocyte lysate”, J. Biol. Chem.; Vol 275(48), pp. 37462-37468.
Hickey E, Brandon S E, Smale G, Lloyd D and Weber L A. 1999 “Sequence and regulation of a gene encoding a human 89-kilodalton heat shock protein”, Mol. Cell. Biol., Vol. 9, pp. 2615-2626.
Hoang A T, Huang J, Rudra-Gonguly N, Zheng J, Powell W C, Rabindron S K, Wu C and Roy-Burman P. 2000 “A novel association between the human heat shock transcription factor I (HSF1) and prostate adenocarcinoma, Am. J. Pathol., Vol. 156, pp. 857-864.
Hostein I, Robertson D, Di Stefano F, Workman P and Clarke P A. 2001 “Inhibition of signal transduction by the HSP90 inhibitor 17-allylamino-17-demethoxygeldanamycin results in cytostasis and apoptosis”, Cancer Res., Vol. 61, pp. 4003-4009.
Hur E, Kim H-H, Choi S M, Kim J H, Yim S, Kwon H J, Choi Y, Kim D K, Lee M-O, Park H. 2002 “Reduction of hypoxia-induced transcription through the repression of hypoxia-inducible factor-1α/aryl hydrocarbon receptor nuclear translocator DNA binding by the 90-kDa heat-shock protein inhibitor radicicol”, Mol. Pharmacol., Vol 62(5), pp. 975-982.
Hutter et al, 1996, Circulation, Vol. 94, pp. 1408.
Jameel A, Skilton R A, Campbell T A, Chander S K, Coombes R C and Luqmani Y A. 1992 “Clinical and biological significance of HSP89a in human breast cancer”, Int. J. Cancer, Vol. 50, pp. 409-415.
Jolly C and Morimoto R I. 2000 “Role of the heat shock response and molecular chaperones in oncogenesis and cell death”, J. Natl. Cancer Inst., Vol. 92, pp. 1564-1572.
Kawanishi K, Shiozaki H, Doki Y, Sakita I, Inoue M, Yano M, Tsujinata T, Shamma A and Monden M. 1999 “Prognostic significance of heat shock proteins 27 and 70 in patients with squamous cell carcinoma of the esophagus”, Cancer, Vol. 85, pp. 1649-1657.
Kelland L R, Abel G, McKeage M J, Jones M, Goddard P M, Valenti M, Murrer B A and Harrap K R. 1993 “Preclinical antitumour evaluation of bis-acetalo-amino-dichloro-cyclohexylamine platinum (IV): an orally active platinum drug”, Cancer Research, Vol. 53, pp. 2581-2586.
Kelland L R, Sharp S Y, Rogers P M, Myers T G and Workman P. 1999 “DT-diaphorase expression and tumor cell sensitivity to 17-allylamino, 17-demethoxygeldanamycin, an inhibitor of heat shock protein 90”, J. Natl. Cancer Inst., Vol. 91, pp. 1940-1949.
Kurebayashi J, Otsuki T, Kurosumi M, Soga S, Akinaga S, Sonoo, H. 2001 “A radicicol derivative, KF58333, inhibits expression of hypoxia-inducible factor-1α and vascular endothelial growth factor, angiogenesis and growth of human breast cancer xenografts”, Jap. J. Cancer Res., Vol 92(12), 1342-1351.
Kwon H J, Yoshida M, Abe K, Horinouchi S and Bepple T. 1992 “Radicicol, an agent inducing the reversal of transformed phentoype of src-transformed fibroblasts, Biosci., Biotechnol., Biochem., Vol. 56, pp. 538-539.
Lebeau J, Le Cholony C, Prosperi M T and Goubin G. 1991 “Constitutive overexpression of 89 kDa heat shock protein gene in the HBL100 mammary cell line converted to a tumorigenic phenotype by the EJ/T24 Harvey-ras oncogene”, Oncogene, Vol. 6, pp. 1125-1132.
Marcu M G, Chadli A, Bouhouche I, Catelli M and Neckers L. 2000a “The heat shock protein 90 antagonist novobiocin interacts with a previously unrecognized ATP-binding domain in the carboxyl terminus of the chaperone”, J. Biol. Chem., Vol. 275, pp. 37181-37186.
Marcu M G, Schulte T W and Neckers L. 2000b “Novobiocin and related coumarins and depletion of heat shock protein 90-dependent signaling proteins”, J. Natl. Cancer Inst., Vol. 92, pp. 242-248.
Martin K J, Kritzman B M, Price L M, Koh B, Kwan C P, Zhang X, MacKay A, O'Hare M J, Kaelin C M, Mutter G L, Pardee A B and Sager R. 2000 “Linking gene expression patterns to therapeutic groups in breast cancer”, Cancer Res., Vol. 60, pp. 2232-2238.
Neckers L, Schulte T W and Momnaaugh E. 1999 “Geldanamycin as a potential anti-cancer agent: its molecular target and biochemical activity”, Invest. New Drugs, Vol. 17, pp. 361-373.
Page J, Heath J, Fulton R, Yalkowsky E, Tabibi E, Tomaszewski J, Smith A and Rodman L. 1997 “Comparison of geldanamycin (NSC-122750) and 17-allylaminogeldanamycin (NSC-330507D) toxicity in rats”, Proc. Am. Assoc. Cancer Res., Vol. 38, pp. 308.
Panaretou B, Prodromou C, Roe S M, O'Brien R, Ladbury J E, Piper P W and Pearl L H. 1998 “ATP binding and hydrolysis are essential to the function of the HSP90 molecular chaperone in vivo”, EMBO J., Vol. 17, pp. 4829-4836.
Plumier et al, 1997, Cell. Stress Chap., Vol. 2, pp. 162
Pratt W B. 1997 “The role of the HSP90-based chaperone system in signal transduction by nuclear receptors and receptors signalling via MAP kinase”, Annu. Rev. Pharmacol. Toxicol., Vol. 37, pp. 297-326.
Prodromou C and Pearl L H. 2000a “Structure and in vivo function of HSP90”, Curr. Opin. Struct. Biol., Vol. 10, pp. 46-51.
Prodromou C, Roe S M, O'Brien R, Ladbury J E, Piper P W and Pearl L H. 1997 “Identification and structural characterization of the ATP/ADP-binding site, in the HSP90 molecular chaperone”, Cell, Vol. 90, pp. 65-75.
Prodromou C, Panaretou B, Chohan S, Siligardi G, O'Brien R, Ladbury J E, Roe S M, Piper P W and Pearl L H. 2000b “The ATPase cycle of HSP90 drives a molecular ‘clamp’ via transient dimerization of the N-terminal domains”, EMBO J., Vol. 19, pp. 4383-4392.
Rajder et al, 2000, Ann. Neurol., Vol. 47, pp. 782.
Roe S M, Prodromou C, O'Brien R, Ladbury J E, Piper P W and Pearl L H. 1999 “Structural basis for inhibition of the HSP90 molecular chaperone by the antitumour antibiotics radicicol and geldanamycin”, J. Med. Chem., Vol. 42, pp. 260-266.
Rutherford S L and Lindquist S. 1998 “HSP90 as a capacitor for morphological evolution. Nature, Vol. 396, pp. 336-342.
Schulte T W, Akinaga S, Murakata T, Agatsuma T, Sugimoto S, Nakano H, Lee Y S, Simen B B, Argon Y, Felts S, Toft D O, Neckers L M and Sharma S V. 1999 “Interaction of radicicol with members of the heat shock protein 90 family of molecular chaperones”, Mol. Endocrinology, Vol. 13, pp. 1435-1448.
Schulte T W, Akinaga S, Soga S, Sullivan W, Sensgard B, Toft D and Neckers L M. 1998 “Antibiotic radicicol binds to the N-terminal domain of HSP90 and shares important biologic activities with geldanamcyin”, Cell Stress and Chaperones, Vol. 3, pp. 100-108.
Schulte T W and Neckers L M. 1998 “The benzoquinone ansamycin 17-allylamino-17-deemthoxygeldanamcyin binds to HSP90 and shares important biologic activities with geldanamycin”, Cancer Chemother. Pharmacol., Vol. 42, pp. 273-279.
Sittler et al, 2001, Hum. Mol. Genet., Vol. 10, pp. 1307.
Smith D F. 2001 “Chaperones in signal transduction”, in: Molecular chaperones in the cell (P Lund, ed.; Oxford University Press, Oxford and NY), pp. 165-178.
Smith D F, Whitesell L and Katsanis E. 1998 “Molecular chaperones: Biology and prospects for pharmacological intervention”, Pharmacological Reviews, Vol. 50, pp. 493-513.
Song H Y, Dunbar J D, Zhang Y X, Guo D and Donner D B. 1995 “Identification of a protein with homology to hsp90 that binds the type 1 tumour necrosis factor receptor”, J. Biol. Chem., Vol. 270, pp. 3574-3581.
Stebbins C E, Russo A, Schneider C, Rosen N, Hartl F U and Pavletich N P. 1997 “Crystal structure of an HSP90-geldanamcyin complex: targeting of a protein chaperone by an antitumor agent”, Cell, Vol. 89, pp. 239-250.
Supko J G, Hickman R L, Grever M R and Malspeis L. 1995 “Preclinical pharmacologic evaluation of geldanamycin as an antitumour agent”, Cancer Chemother. Pharmacol., Vol. 36, pp. 305-315.
Tratzelt et al, 1995, Proc. Nat. Acad. Sci., Vol. 92, pp. 2944.
Trost et al, 1998, J. Clin. Invest., Vol. 101, pp. 855.
Tytell M and Hooper P L. 2001 “Heat shock proteins: new keys to the development of cytoprotective therapies”, Emerging Therapeutic Targets, Vol. 5, pp. 267-287.
Uehara U, Hori M, Takeuchi T and Umezawa H. 1986 “Phenotypic change from transformed to normal induced by benzoquinoid ansamycins accompanies inactivation of p60src in rat kidney cells infected with Rous sarcoma virus”, Mol. Cell. Biol., Vol. 6, pp. 2198-2206.
Waxman, Lloyd H. Inhibiting hepatitis C virus processing and replication. (Merck & Co., Inc., USA). PCT Int. Appl. (2002), WO 0207761
Winklhofer et al, 2001, J. Biol. Chem., Vol. 276, 45160.
Whitesell L, Mimnaugh E G, De Costa B, Myers C E and Neckers L M. 1994 “Inhibition of heat shock protein HSP90-pp 60v-src heteroprotein complex formation by benzoquinone ansamycins: essential role for stress proteins in oncogenic transformation”, Proc. Natl. Acad. Sci. USA., Vol. 91, pp. 8324-8328.
Yorgin et al. 2000 “Effects of geldanamycin, a heat-shock protein 90-binding agent, on T cell function and T cell nonreceptor protein tyrosine kinases”, J. Immunol., Vol 164(6), pp. 2915-2923.
Young J C, Moarefi I and Hartl F U. 2001 “HSP90: a specialized but essential protein-folding tool”, J. Cell. Biol., Vol. 154, pp. 267-273.
Zhao J F, Nakano H and Sharma S. 1995 “Suppression of RAS and MOS transformation by radicicol”, Oncogene, Vol. 11, pp. 161-173.

1. A compound of formula (I), or a salt, N-oxide, hydrate, or solvate thereof: 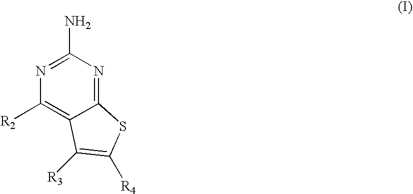
wherein
R₂ is a group of formula (IA):
-(Ar¹)ₘ-(Alk¹)ₚ-(Z)ᵣ-(Alk²)ₛ-Q (IA)
wherein in any compatible combination
Ar¹ is an optionally substituted aryl or heteroaryl radical,
Alk¹ and Alk² are optionally substituted divalent C₁-C₃ alkylene or C₂-C₃ alkenylene radicals,
m, p, r and s are independently 0 or 1,
Z is —O—, —S—, —(C═O)—, —(C═S)—, —SO₂—, —C(═O)O—, —C(═O)NR^{A}—, —C(═S)NR^{A}—, —SO₂NR^{A}—, —NR^{A}C(═O)—, —NR^{A}SO₂— or —NR^{A}— wherein R^{A} is hydrogen or C₁-C₆ alkyl, and
Q is hydrogen or an optionally substituted carbocyclic or heterocyclic radical;
R₃ is hydrogen, an optional substituent, or an optionally substituted (C₁-C₆)alkyl, aryl or heteroaryl radical; and
R₄ is
(i) hydrogen, a —CN group, a nitro group —NO₂, or a —C(═NOH)(NH₂) group, or
(ii) an optionally substituted C₁-C₆alkyl, aryl, heterocyclic, aryl(C₁-C₆alkyl)-, or heterocyclic(C₁-C₆alkyl)- group, or
(iii) a group of formula —C(═O)R₅ wherein R₅ is hydroxyl, optionally substituted C₁-C₆alkyl, C₁-C₆alkyoxy, aryl, aryloxy, heteroaryl, heteroaryloxy, aryl(C₁-C₆alkyl)-, aryl(C₁-C₆alkoxy)-, heteroaryl(C₁-C₆alkyl)-, or heteroaryl(C₁-C₆alkoxy)-, or
(iv) a group of formula —C(═O)NHR₆ wherein R₆ is primary, secondary, tertiary or cyclic amino, or hydroxyl, optionally substituted C₁-C₆alkyl, C₁-C₆alkyoxy, aryl, aryloxy, heteroaryl, heteroaryloxy, aryl(C₁-C₆alkyl)-, aryl(C₁-C₆alkoxy)-, heteroaryl(C₁-C₆alkyl)-, or heteroaryl(C₁-C₆alkoxy)-.

2. A compound as claimed in claim 1 wherein R₄ is a —CN group, or an optionally substituted C₁-C₆alkyl, aryl, heteroaryl, aryl(C₁-C₆alkyl)-, or heteroaryl(C₁-C₆alkyl)- group.

3. A compound as claimed in claim 2 wherein R₄ is
(a) an imidazolyl or oxadiazolyl group, a C₁-C₆alkyl group, optionally substituted by a hydroxyl or primary, secondary, tertiary or cyclic amino group, or
(b) a group of formula —C(═O)R₅ wherein R₅ is C₁-C₆alkyl or phenyl, or
(c) a group of formula —C(—O)NHR₆ wherein R₆ is N-piperidinyl, N-morpholinyl, N-piperazinyl, N¹-methyl-N-piperazinyl, N-triazolyl, C₁-C₆alkyoxy, or mono or di-C₁-C₆alkylamino.

4. A compound as claimed in claim 1 wherein R₄ is an optionally substituted phenyl, phenyl(C₁-C₆alkyl)-, heterocyclic or heterocyclic(C₁-C₆alkyl)- group wherein the heterocyclic part is monocyclic with 5 or 6 ring atoms.

5. A compound as claimed in claim 4 wherein R₄ is an optionally oxadiazolyl, imidazolyl, dihydro-imidazolyl, triazolyl, pyrazolyl, pyrrolyl, thiazolyl or tetrazolyl group

6. A compound as claimed in claim 4 wherein R₄ is an oxadiazol-3-yl, 4,5-dihydro-1H-imidazol-2-yl, [1,2,4]triazol-4-yl, 5-amino-1H-[1,2,4]triazol-3-yl, 4- or 5-methyl-2H-pyrazol-3-yl, 1H-pyrrol-2-yl, 2-amino-5-methyl-thiazol-4-yl, 3H-imidazol-4-yl, or 2H-tetrazol-5-yl group.

7. A compound as claimed in claim 1 wherein R₄ is optionally substituted methyl, ethyl or n-propyl.

8. A compound as claimed in claim 7 wherein substituents in R₄ are selected from amino, methylamino, ethylamino, n-propylamino, acetamido, oxo, hydroxyl, phenyl, methyl, ethyl, and n-propyl.

9. A compound as claimed in claim 7 wherein R₄ is acetamidomethyl, formyl, 2-hydroxy-2-methyl-propyl, 2-hydroxy-2-ethyl-but-1-yl, hydroxymethyl, ethylcarbonyl, phenylcarbonyl, n-propylaminomethyl, aminomethyl, or diphenyl-hydroxymethyl,

10. A compound as claimed in claim 1 wherein, in the group R₂, m is 1, each of p, r and s is 0, and Q is hydrogen.

11. A compound as claimed in claim 1 wherein R₂ is optionally substituted phenyl, 2- or 3-thienyl, 2- or 3-furanyl, 2-, 3- or 4-pyridinyl, morpholinyl, or piperidinyl.

12. A compound as claimed in claim 11 wherein, in the compound (I), R₂ is phenyl, optionally substituted by a one or more substituents selected from methyl, ethyl, n- or isopropyl, vinyl, allyl, methoxy, ethoxy, n-propyloxy, benzyloxy, allyloxy, cyanomethoxy, chloro, bromo, cyano, formyl, methyl-, ethyl-, or n-propyl-carbonyloxy, methyl- or ethylaminocarbonyl, and substituents of formula —O(CH₂)ₙZ¹ wherein n is 1, 2 or 3 and Z¹ is a primary, secondary, tertiary or cyclic amino group, or a C₁-C₆alkoxy group; or of formula -(Alk³)ₘZ¹ wherein Alk³ is a divalent straight or branched chain (C₁-C₃) alkylene, m is 0 or 1, and Z¹ is a primary, secondary, tertiary or cyclic amino group, or a C₁-C₆alkoxy group.

13. A compound as claimed in claim 12 wherein optional substituents are in the 2- and/or 4- and/or 5-position of the phenyl ring.

14. A compound as claimed in claim 1, wherein, in the group R₂, m is 1, and p, r and s are 0, and Q is an optionally substituted carbocyclic or heterocyclic ring.

15. A compound as claimed in claim 1 which has formula (II): 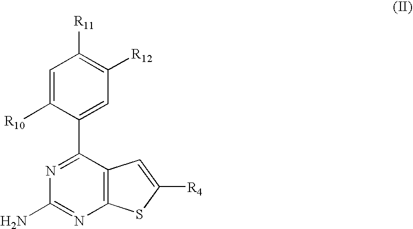
wherein
R₄ is as defined in any of claims 1 to 9;
R₁₀ is H, Cl, Br, or CH₃;
R₁₁ is hydrogen, Cl, Br, CN, methyl, ethyl, n- or iso-propyl, vinyl or allyl;
R₁₂ is (i) a radical of formula —O(CH₂)ₙZ¹ wherein n is 1, 2 or 3 and Z¹ is a primary, secondary, tertiary or cyclic amino group, or a C₁-C₆alkoxy group; or (ii) a radical of formula -(Alk³)ₘZ¹ wherein Alk³ is a divalent straight or branched chain (C₁-C₃) alkylene, m is 0 or 1, and Z¹ is a primary, secondary, tertiary or cyclic amino group, or a C₁-C₆alkoxy group.

16. A pharmaceutical or veterinary composition comprising a compound as claimed in claim 1, together with a pharmaceutically or veterinarily acceptable carrier.

17. A composition comprising a compound as claimed in claim 1 in in an amount effective to inhibit HSP90 activity in vitro or in vivo.

18. A method of treatment of diseases or conditions which are responsive to inhibition of HSP90 activity in mammals which method comprises administering to the mammal an amount of a compound as claimed in claim 1 effective to inhibit said HSP90 activity.

19. The method as claimed claim 18 for immunosuppression or the treatment of cancer; viral disease, rheumatoid arthritis, asthma, multiple sclerosis, Type I diabetes, lupus, psoriasis and inflammatory bowel disease; cystic fibrosis angiogenesis-related disease such as diabetic retinopathy, haemangiomas, and endometriosis; or for protection of normal cells against chemotherapy-induced toxicity; or diseases where failure to undergo apoptosis is an underlying factor; or protection from hypoxia-ischemic injury due to elevation of Hsp70 in the heart and brain; scrapie/CJD, Huntingdon's or Alzheimer's disease.
